SEQUENCE LISTING (7 sequences)

SEQ ID NO: 1 - protein, 8 aa, Artificial Sequence
  Description of Artificial Sequence Xpress Leader peptide
DLYDDDDK

SEQ ID NO: 2 - protein, 11 aa, Homo sapiens
EQKLISEEDLN

SEQ ID NO: 3 - protein, 14 aa, Homo sapiens
GKPIPNPLLGLDSY

SEQ ID NO: 4 - protein, 8 aa, Paramoxyvirus PV protein
DYKDDDDK

SEQ ID NO: 5 - protein, 5 aa, Artificial Sequence
  Description of Artificial Sequence  Factor Xa recognition sequence
IEGRG

SEQ ID NO: 6 - protein, 5 aa, Artificial Sequence
  Description of Artificial Sequence Enterokinase recognition sequence
DDDDK

SEQ ID NO: 7 - protein, 4 aa, Artificial Sequence
  Description of Artificial Sequence Factor Xa recognition sequence
IEGR

(19) United States
(12) Patent Application Publication
(10) Pub. No.: US 2005/0255464 A1
(43) Pub. Date: Nov. 17, 2005

(54) Methods for the identification of peptidyl compounds interacting with extracellular target molecules

(76) Inventors: Frederick S. Hagen, Seattle, WA (US); Richard G. Woodbury, Seattle, WA (US); Pieter J. Oort, Shoreline, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/484,271

(22) PCT Filed: Jul. 18, 2002
(86) PCT No.: PCT/US02/23375

§ 371 Date: Jan. 20, 2004

Related U.S. Application Data
(60) Provisional application No. 60/306,924, filed on Jul. 19, 2001.

(51) Int. Cl. C12Q 1/68; G01N 33/53; C12N 15/85

(52) U.S. Cl. 435/6; 435/7.1; 435/455; 435/325

(57) ABSTRACT

The present invention provides libraries expressing peptide libraries on the extracellular cell surface of host cells and methods for identifying peptides that bind extracellular target molecules under the physiological conditions encountered in biological fluids and secretions. The present invention is also directed to vectors for expressing gene fusion proteins and for targeting those fusion proteins to the extracellular cell surface.

### CROSS-REFERENCE TO RELATED APPLICATIONS

This application claims the benefit of U.S. Patent application Ser. No. 60/306,924, filed Jul. 19, 2001, the disclosure of which is incorporated herein by reference.

### BACKGROUND OF THE INVENTION

Intercellular function is mediated by protein interactions with macromolecules in the extracellular space. For example, cell-surface proteins and soluble proteins secreted by cells bind extracellularly to their cognate ligands, receptors, enzymatic substrates, or other extracellular macromolecules to initiate intracellular signaling cascades, release cell-surface proteins from the plasma membrane, localization of cells to target sites, or to produce other changes in the extracellular environment. Such proteins include, for example, antibodies that mediate immune responses; cytokines or chemokines that regulate diverse cell growth, differentiation, or cell death pathways; enzymes of the major blood cascade pathways; and the like. Because these responses are involved in normal cell function, inappropriate induction, disruption, or stabilization of extracellular protein interactions play a key role in disease pathogenesis.

Screening methods have been developed to identify peptides that affect cellular processes through perturbation of protein interactions. These methods involve the development of large peptide libraries, such as phage display libraries (see, e.g., Scott and Smith, Science 249:386-90 (1990)), combinatorial libraries, peptide mimetic libraries, and one-bead-one structure combinatorial libraries. (See, e.g., al-Obeidi et al., Mol. Biotechnol. 9:205-23 (1998).) Libraries of peptides can be used in in vitro screening assays to identify those peptides that specifically interact with proteins or other macromolecules.

Conventional peptide screens suffer from various disadvantages, however, that limit the efficient identification of therapeutically promising peptides, particularly peptides that act within the extracellular space. Most of the screening methods that employ conventional peptide libraries demonstrate only binding to targets in vitro; these methods often fail to identify peptides that bind to extracellular targets with corresponding physiological effects in vivo. Subsequent biological screens of the peptides are required to identify those peptides with an appropriate effect on target protein function in vivo. Such methods are not optimized to identify therapeutically promising peptides because they are ineffective in initial screens in discriminating between weak and strong interactions, and between specific and non-specific binding variants. Also, these methods can screen out promising peptides that demonstrate a relatively weak affinity and yet induce an appropriate physiological response. Such methods also rely upon further costly and time-consuming experimentation to identify effectors of protein activity.

Current in vitro screening methods also suffer from the disadvantage that the normal structure, activity, and any necessary regulatory molecule(s) may be lost when proteins or other macromolecules are purified or removed from their native environment. Peptides that would normally be effector molecules of native macromolecules may not bind to structurally altered targets. Non-native targets are also more likely than native targets to non-specifically bind physiologically irrelevant peptides. Further, even if purified target molecules retain native structure and activity, existing screening methods can produce poor or misleading results because the assay conditions are not representative of the local extracellular environment in vivo. Local environments can significantly influence the accessibility of target molecules to peptides or the specificity or avidity of peptide binding.

Conventional screening methods also typically utilize target molecules that are attached to non-physiological surfaces, such as plastic, glass, or polymeric matrices. This association with a non-physiological surface introduces impediments to identifying peptides that interact specifically with protein or other macromolecular target molecules. Many macromolecular target molecules that are attached to a non-physiological surface denature onto that surface. Native binding sites on the surface of targets can be lost and other sites not normally displayed on the surface of targets can be unmasked, exposing such physiologically irrelevant sites to the peptides. This problem can result in the identification of peptides that only bind non-specifically to targets. The mode of attachment can also bias how target molecules are exposed on the surface and can result in a spatial orientation where only one set, or a limited set, of potential binding sites are exposed to the peptides. When this occurs, functionally important peptide binding sites on targets can be inaccessible during screening. Another impediment is that the binding kinetics and constants of freely soluble, interacting molecules can be altered when one is attached directly to a non-physiological surface. This problem is a well-known phenomenon that can either increase or decrease the specificity and avidity of peptide binding to targets and can lead to the identification of peptides that are ineffective in subsequent, functional screens (see, e.g., Vijayendran and Leckband, Anal. Chem. 73:471-80 (2001); Butler, Methods 22:4-23 (2000)).

Further, chemical-based combinatorial peptide libraries, consisting of small peptides that are not attached to a soluble carrier molecule or a hydrophilic matrix, suffer from the disadvantage that many short peptides are not soluble under physiological conditions, such as in the presence of undiluted blood, plasma, serum, or other complex biological fluids. Organic solvents such as methanol, ethanol, or DMSO have been required in prior screens to maintain the solubility of many peptides in the library. These organic solvents can denature many potential target or non-target proteins and other macromolecules during screening and result in the identification of poor-quality peptide candidates.

An additional disadvantage experienced with methods using phage- and bacteria-display peptide libraries is the prevalence of high backgrounds due to nonspecific binding of phage or bacteria to the targets. Such background can occur when screening is conducted in physiologic environments, thereby causing many irrelevant peptide candidates to be selected. Typically, the nonspecific binding of phage and bacteria can be reduced by screening in the presence of high concentrations of salt, denaturants (e.g., urea or guanidine-HCl), protein, or detergent, or other non-physiological conditions (e.g., elevated temperatures, such as above 37° C.). In contrast, physiological screening conditions for the identification of peptides usually replicate the conditions in which the target molecules normally express their activities (e.g., human blood at 37° C.). However, the complexity of macromolecules (e.g., blood) present under physiological conditions can lead to a high level of nonspecific binding of peptide-displaying phage or bacteria, such that the library's diversity can be significantly reduced.

Some screening methods have sought to address these limitations, but largely with respect to intracellular target molecules. For example, methods have been developed to screen small peptides and polypeptides intracellularly. Such methods typically utilize intracellular expression of peptides or polypeptides as fusion products, such as in the yeast two hybrid system. (See Fields and Song, Nature 340:245-46 (1989).) Other methods can present peptides and small proteins in vivo in constrained configurations on carrier proteins or displayed within a reporter protein. (See, e.g., Cull et al., Proc. Natl. Acad. Sci. USA 89:1865-69 (1992); Lu et al., Biotechnology (NY)13:366-72 (1995); International Patent Publication WO 99/24617; Norman et al., Science 285:591-95 (1999); International Patent Publication WO 98/39483.) For example, International Patent Publication WO 99/24617 discloses peptides displayed as a fusion with green fluorescent protein (“GFP”). In addition, some methods allow identification of small peptides and polypeptides based on their ability, when expressed intracellularly, to alter cell function through perturbation of cellular protein interactions. (See, e.g., International Patent Application WO 98/07886; Caponigro et al., Proc. Natl. Acad. Sci. USA 95:7508-13 (1998).) However, these methods, while preserving the native, cytosolic constituents of intracellular pathways, do not allow for the screening of extracellular target molecules under relevant physiological conditions.

Other methods have been described that allow peptide library sequences to be expressed extracellularly on eukaryotic cells, including mammalian and other animal cells (see U.S. Pat. No. 6,153,380; International Patent Application WO 98/39483). These methods still do not allow peptide interactions to be screened under physiological conditions, such as in the presence of undiluted blood, plasma, serum, or other complex biological fluids. For example, one method allows randomized peptides to be inserted into host cells and depending on the fusion construct used, either localized to the extracellular or intracellular cell surface or secreted in soluble form. (See U.S. Pat. No. 6,153,380.) Peptides can then be selected by assaying for their ability to alter the phenotype of either the host cells or, alternatively, of another cell population. While allowing for the identification of peptides that affect extracellular interactions, this method does not address the need for assay conditions that reproduce the target molecule's native physiological conditions. Such conditions, including those that affect molecular conformation, stability, binding kinetics, and the like, can be significant for maintaining normal interactions of the target molecule with other extracellular macromolecules.

Analysis of the human genome indicates approximately 1.6% of the gene products are proteases (Southan, FEBS Lett. 498:214-218 (2001)). Therefore, humans produce between 400 to 700 different proteases. Approximately 350 unique human protease mRNAs are found in GenBank, indicating that as many as one-half of all human proteases have not been studied (Southan, J. Pept. Sci. 6:453-458 (2000)). Further, 14% of all known human proteases currently are targets of drug development (Southan, Drug Discovery Today 6:681-688 (2001)). The physiologic functions and pathologic activities of many proteases, even those previously studied, are unknown. Thus, while proteases are important in many biologic processes including fertilization, cellular differentiation, cellular regulation, inflammation, blood coagulation, fibrinolysis, tissue remodeling/repair, host defense systems against pathogens, cancer, programmed cell death and others, the functions of many human proteases are unknown.

There is increasing awareness of the potential of protease inhibitors as clinical drugs, stimulated to a large extent by the success of protease inhibitors in HIV treatment. A major impediment in the identification of clinically applicable protease inhibitors has been the absence of a high throughput screening technology capable of identification of inhibitors under physiological conditions. The use of the invention described herein is expected to facilitate the identification of biologically relevant inhibitors of target proteases. Targeted proteases and extracellular displayed peptides can be combined under near physiological conditions to identify the detailed peptide substrate specificity of proteases that may lead to the development of selective inhibitors of the proteases. Alternatively, certain constrained “loop” peptides may act directly as protease inhibitors.

Proteases have specific substrate amino acid sequence requirements for physiologic activity. A system of nomenclature to describe protease-substrate interaction was implemented by Schechter and Berger (Biochem. Biophys. Res. Commun. 27:157-162 (1967)). The bond cleaved is designated:
P₁-P₁′
For proteases that cleave many different peptides and proteins, e.g., trypsin, chymotrypsin, and subtilisin, the nature of the P₁ amino acid residue is the most important specificity element. Proteases involved in regulatory pathways typically have substrates with more complex specificity information. For some proteases, physiologic substrates contain an accessible, unique amino acid sequence defined by as many as 5 or 6 consecutive amino acid residue positions such as:
P₅-P₄-P₃-P₂-P₁ - - - P₁′
An example is enterokinase that cleaves proteins or peptides containing the amino acid residue sequence:
Asp-Asp-Asp-Asp-Lys - - - P₁′ (SEQ. ID NO.: 6)
Another example is Factor Xₐ that selectively cleaves peptides or proteins containing the following sequence:
Ile-Glu-Gly-Arg - - - P₁′ (SEQ ID NO.: 7)

Some proteases require substrates with specific amino acid residues at the amino terminal side of hydrolyzed bonds, i.e., at P₁′-P₂′-P₃′ etc. positions. An example of this is the family of aspartic acid proteases.

There are corresponding binding sites or “pockets” designated as, e.g., S₃-S₂-S₁, on the surface of the protease molecule to accommodate the side chains of each amino acid residue contributing to specificity. Occasionally, one or a few conservative amino acid substitutions in preferred peptide substrates can be made without significant loss in protease activity.

Conventional approaches for determining the substrate specificity of a protease is a difficult, time-consuming and costly procedure, particularly when the physiologic substrate is unknown. Frequently, only a general or partial specificity map is possible. Two conventional approaches have been used to determine protease specificity. The first comprises analytical characterization of peptide products following protease digestion of reduced and denatured proteins of known amino acid sequences, e.g., insulin, ribonuclease, and others (Le Trong et al., Proc. Natl. Acad. Sci. USA 84:364-367 (1987)). This approach provides only a simple description of specificity, concluding, e.g., that a protease has “trypsin-like” activity.

The second method comprises the use of synthetic peptide substrates containing a detectable leaving group, e.g., p-nitroaniline that is measured when an appropriate bond is cleaved (Yoshida et al., Biochemistry 19:799-804 (1980)). This tedious, costly approach involves extensive trial and error effort. For example, the determination of a protease's specificity requiring only two amino acid residues, i.e., P₂-P₁ could require testing as many as 400 different synthetic substrates. A 3 amino acid residue specificity sequence (P₃-P₂-P₁) may involve testing thousands of substrates. A further complication with this approach is that many short peptide substrates are insoluble under conditions required for optimum protease activity and their solubility may require concentrations of organic solvents that denature the protease or alter enzyme activity.

The presentation of random peptide libraries by phage display is a useful approach to search for peptides with biologic activities (Roberge et al., Biochemistry 40:9522-9531 (2001); Kridel et al., Anal. Biochem. 294:176-184 (2001); and Leinonen et al., Scand. J. Clin. Lab. Invest. Suppl. 233:59-64 (2000)). Matthews and co-workers (U.S. Pat. No. 5,846,765) reported a phage display system to search for suitable substrate peptides of a mutant variant of subtilisin. The system displays a fusion protein consisting of phage coat protein attached to the substrate peptides attached to a polypeptide “reporter” that can be bound by specific antibody, receptor or other means. Phage that display appropriate substrate peptides lose the reporter group and can be selected from inappropriate ones via affinity adsorption. Alternatively, the phage can be pre-adsorbed to an affinity surface, incubated with protease and appropriate substrate peptides selected from among those phage particles released from the surface. These investigators suggest that this approach could be used to determine the substrate specificity of proteases in general. In practice, however, the phage peptide display approach may be limited to relatively “robust” proteases that retain activity under non-physiologic screening conditions (e.g., high concentrations of salt, inclusion of detergents and/or EDTA) required to prevent undesired adsorption of phage particles. Also, screening under non-physiologic conditions may alter normal protease specificity and other enzyme properties.

Additionally, several reports have emphasized the technical difficulties in implementing polyvalent display phage methods that frequently result in preparations containing mixtures of peptides where the expression level is low, the quality of the peptides is inconsistent and screening is unreliable (see U.S. Pat. No. 5,846,765). Many phage-based screening methods require selection of desired phage from inappropriate ones by affinity adsorption, e.g., with specific antibodies. In other screens selection is based on direct interaction of the macromolecule of interest with peptides displayed on phage. Selection of phage particles by affinity adsorption methods frequently is limited by the inability to distinguish between phage products with high binding affinities from those with low affinities (Cwirla et al., Proc. Nat. Acad. Sci. USA 87:6372-6382 (1990)). The inability to distinguish between peptides with high and low binding affinities in screens results in many false positives. A similar outcome may occur when phage peptide libraries are screened for substrates and inhibitors under conditions of low stringency required for optimal protease activity. Therefore, while phage display methods are innovative approaches, they may result in a high number of poor quality prospects that must be systematically culled from promising ones.

There is need in drug development for rapid, more efficient screening approaches, conducted under optimum physiologic conditions, to identify substrate peptides of human proteases, particularly poorly characterized and “orphan” enzymes. Therefore, there remains a need for screening methods that will allow for more efficient identification of peptides that bind to extracellular target molecules by replicating the complex physiological conditions of the extracellular environment. Because screening under physiological conditions is more likely to reduce non-specific binding events, retain native molecular conformations and kinetics, and maintain the presence of regulatory molecules, such physiologically-based screens are more likely to identify molecular interactions associated with normal or disease-related cellular function. Consequently, the development of robust technologies to express peptide libraries of high diversity on the surface of mammalian cells and that allow peptide screening under physiological conditions are needed to facilitate the identification of more effective drugs and therapeutic approaches to disease.

### SUMMARY OF THE INVENTION

The present invention generally relates to methods for identifying peptides that specifically bind to extracellular target molecules under physiological or substantially physiological conditions. In one aspect, the methods include introducing an expression library into mammalian host cell. The library includes a plurality of oligonucleotides. At least a majority of the oligonucleotides in the library have different sequences encoding different peptides. The host cells express and display the peptides on an extracellular cell surface. The host cells displaying the peptides are contacted with at least one extracellular target molecule under substantially physiological conditions. A first subset of host cells that bind to the target molecule is selected from the first plurality of host cells. A first sub-library (of the expression library) is recovered from the first subset of host cells, the first sub-library including at least one oligonucleotide that encodes a peptide that binds to the target molecule(s).

The target molecule can be contacted with the host cells in the presence of a complex biological fluid. Suitable complex biological fluids include, for example, blood, serum, plasma, sweat, tears, urine, semen, vaginal fluid, mucous, and the like. In one embodiment, the oligonucleotides have a length of about 18 to about 60 nucleotides. In another embodiment, the oligonucleotides encode peptides having a length of about 6 to about 20 amino acid residues. The oligonucleotides can optionally have randomized or semi-randomized sequences.

For further screening, the first sub-library is introduced into a second plurality of host cells, the second plurality of host cells expressing peptides encoded by the first sub-library and displaying the peptides on the extracellular cell surface. The second plurality of host cells is contacted with the at least one extracellular target molecule, and a second subset of host cells is selected that display peptides that bind to the target molecule. A second sub-library is recovered from the second subset of selected host cells, the second sub-library including at least one oligonucleotide that encodes the peptide that binds to the target molecule(s).

In certain embodiments, the host cells express and display a high copy number of the peptides on the extracellular cell surface. The target molecule can be an extracellular protein, such as, for example, a peptide, protein, a tumor-specific antigen, a tumor-associated antigen, an antibody, a glycoprotein, a phosphoprotein, a glycophosphoprotein, a proteoglycan, a carbohydrate, a lipid, or a polymeric complex thereof. The target molecule is displayed on an extracellular surface of a target cell or can be soluble in a complex biological fluid. The target cell can be an animal cell, a mammalian cell, a bacterial cell, a fungal cell, or the target molecule can be on a virus, phage, parasite, isolated subcellular organelle, and the like. The target molecule also can be a protein associated with an autosomal dominant disease, an oncogenic disease, or with normal cellular function.

The binding of the peptide to the target molecule can result in a change in a detectable phenotype of the target cell. Such a change in a detectable phenotype can result from, for example, altering a function of a mutant protein, an alleviation of factor-dependent growth, a change in apoptotic state of the target cell, and the like.

In certain embodiments, the first subset of host cells is selected by contacting animal cells with a detectably labeled antibody that binds to a marker on the target cells and detecting the bound, labeled antibody. In other embodiments, the first subset of host cells is selected by contacting the host cells with a detectably labeled antibody that binds a marker on the host cells and detecting the bound-labeled antibody. Alternatively, the first subset of host cells can be selected by contacting bacterial cells, fungal cells, viruses, phage, parasites, or isolated subcellular organelles with a detectably labeled antibody that binds a marker on the bacterial cells, fungal cells, viruses, phage, parasites, or isolated subcellular organelles and detecting the bound labeled antibody.

In an embodiment, the target molecule can be, for example, a cell surface receptor, such as a cytokine, a chemokine, a secreted factor, and the like. The peptide can bind to the target molecule, thereby altering (e.g., increasing or decreasing) binding of the target molecule to its cell surface receptor. In another embodiment, the target molecule can be an antibody, and the peptide binds to the antibody, thereby reducing binding of the antibody to an antigen. The antibody can be, for example, an autoantibody.

The peptide can be an extracellular enzyme or protease and peptide binding to the enzyme or protease altering the activity of the enzyme or protease. For example, the peptide can bind to an active site of the enzyme or protease, thereby inhibiting activity of the enzyme or protease. The peptide can be a competitive inhibitor of substrate binding to the enzyme or protease. The peptide can also bind to an allosteric regulatory site on the enzyme or protease. In a particular embodiment wherein the enzyme is a protease the peptide can be a substrate of the protease resulting in cleavage of the peptide. A selectable marker placed at the amino-terminus of peptides tethered to a presenting cell will be released from the cell when the protease cleaves a displayed peptide.

The target molecule can be displayed on an extracellular surface of a target cell. The target molecule can be a mutant protein, and binding of the peptide alters a function of the mutant protein. Alternatively, the target molecule can cause other alterations of function, such as, for example, alleviating factor-dependent growth, changing an apoptotic state of the target cell, increasing or decreasing sensitivity to a cytotoxic drug, and the like.

In certain embodiments, the peptides can be displayed as fusion proteins with a presentation molecule. Suitable presentation molecules include, for example, CD24 or Interleukin 3 receptor. The fusion protein can optionally further include an epitope, such as, for example, polyhistidine, V5, FLAG, or myc. The fusion protein can also optionally include a signal for glycophosphatidylinositol anchorage or a transmembrane domain.

In certain embodiments, the peptides can be displayed as a fusion proteins consisting of thioredoxin and one or two tag elements in addition to the random peptide sequence. For example, a protease substrate random peptide sequence is positioned at the amino-terminal end of thioredoxin between two markers, FLAG and V5, with FLAG as the amino-terminus of the fusion protein. In a particular embodiment, the screen for protease susceptible sequences selects cells lacking the amino terminal FLAG marker due to protease activity that cleaves peptides in the library. Plasmids from selected cells are recovered, amplified in bacteria, reintroduced into naïve cells, the cells are incubated with protease, and screening selects an enriched population of candidates as compared to the first round results. The process is repeated for several rounds until a relatively restricted family of related peptides is obtained. DNA sequence analysis of the selected clones establishes the substrate preference of the selected protease. In a particular embodiment, a specific amino acid sequence, e.g., Ile-Glu-Gly-Arg-X (SEQ ID NO: 7), that is a restricted substrate of the protease Factor Xₐ is inserted at the random peptide site. In certain embodiments, the first subset of host cells is selected by using a flow sorter to identify the first subset of cells exhibiting binding to the target molecule. Alternatively, the target cells can be coupled to magnetic beads; the first subset of host cells can be selected by collecting host cells bound to the target cells on the beads.

In other embodiments, the target molecule is detectably labeled and the first subset of host cells is selected by identifying cells bound to the labeled target molecule. For example, the target molecule can be a detectably labeled antibody, and the first subset of host cells is selected by identifying host cells bound to the labeled antibody.

In another aspect a peptide display library is provided, the peptide library including a plurality of at least one type of expression vector. Each expression vector has a first nucleic acid sequence encoding a signal sequence, a presentation molecule, a transmembrane domain, and a cloning site for insertion of a second nucleic acid sequence distal to the transmembrane domain, the second nucleic acid sequence encoding an amino acid sequence. The encoded fusion protein(s) is expressed and displayed on an extracellular surface of a host cell. The presentation molecule can encode, for example, modified CD24, modified IL-3 receptor, and the like. The second nucleic acid optionally can encode peptides having up to 20 amino acids.

In yet another aspect, a library peptide displayed as an extracellular membrane protein fusion is provided. The library peptide includes modified CD24 having a signal sequence and a transmembrane domain. The library peptide is inserted within the CD24 amino acid sequence. The peptide is displayed on an extracellular cell surface such that the peptide can interact with extracellular target molecules under substantially physiological conditions.

In another aspect, a plasmid expression vector is provided. The expression vector includes an SV40 origin of replication and a nucleic acid sequence including an SV40 early promoter region and an SV40 Large T antigen coding region, the SV40 early region promoter promoting transcription of the Large T antigen coding region; the replication of the plasmid is self-regulating.

In another aspect, a method is provided for identifying peptides that specifically bind to extracellular target molecules. The method generally includes introducing an expression library into a first plurality of mammalian host cells. The expression library has a plurality of oligonucleotides, at least a majority of the oligonucleotides having different sequences encoding different peptides. The host cells express and display the peptides on an extracellular cell surface. The host cells displaying the peptides are contacted with at least one extracellular target molecule in a substantially undiluted complex biological fluid. A first subset of cells displaying peptides that bind to the target molecule is selected, and a first sub-library of the expression library is recovered from the first subset of host cells. The first sub-library includes at least one oligonucleotide that encodes a peptide that binds to the target molecule. The substantially undiluted complex biological fluid can be, for example, blood, plasma, serum, a tissue secretion, sweat, tears, vaginal fluid, mucous, seminal fluid, urine, and the like.

In yet another aspect, a method is provided for identifying peptides that specifically bind to extracellular target molecules. The method generally includes introducing an expression library into a first plurality of host cells. The expression library includes a plurality of oligonucleofides, at least a majority of the oligonucleotides having different sequences encoding different peptides. The host cells express and display the peptides on an extracellular cell surface of the host cells. The host cells are contacted with at least one extracellular target molecule in a complex biological fluid. A first subset of host cells is selected that display peptides that bind to target molecules. A first sub-library is recovered from the first subset of host cells, the first sub-library including at least one oligonucleotide that encodes a peptide that binds to the target molecule(s). The target molecule can be displayed on the extracellular surface of host or non-host animal cells, on bacterial cells, fungal cells, viruses, phage, parasites, isolated subcellular organelles, and the like. In an embodiment, the complex biological fluid can be substantially undiluted.

A further understanding of the nature and advantages of the invention will become apparent by reference to the remaining portions of the specification.

### BRIEF DESCRIPTION OF THE DRAWING

FIG. 1 depicts a representation of one example of an expression vector, designated plcoDual, encoding a CD24 V5 fusion protein and a thioredoxin-FLAG fusion protein, which is suitable for use in the present invention.

FIG. 2 depicts a representation of an expression vector designated pIcoFLAGXa, encoding a CD24 V5 fusion protein and a thioredoxin-FLAG fusion protein where the Factor Xₐ restriction cleavage amino acid sequence, Ile-Glu-Gly-Arg-X (SEQ ID NO: 7), has been inserted at the random peptide site.

### DESCRIPTION OF THE SPECIFIC EMBODIMENTS

Prior to setting forth the invention in more detail, it may be helpful to a further understanding thereof to set forth definitions of certain terms as used hereinafter.

Definitions

The terms “genetic library” or “library” are used interchangeably and refer to a collection of nucleic acid fragments that can individually range in size from about a few base pairs to about a million base pairs. Typically, as used in the context of the present invention, a genetic library comprises random or semi-random oligonucleotides that encode peptides or polypeptides. The oligonucleotides can have an average length of, for example, from about 10 bases to about 60 bases. In certain embodiments, a library is contained as inserts in a vector capable of propagating in certain host cells, such as bacterial, fungal, plant, insect, animal and/or mammalian cells.

The term “sub-library” refers to a portion of a genetic library that has been isolated by methods according to the present invention.

The term “insert” in the context of a library refers to an individual nucleic acid fragment that is typically inserted into single vector (e.g., an expression vector) or an expression construct.

The term “coverage” in the context of a genetic library refers to the amount of redundancy of the library. It will be appreciated by those skilled in the art that the redundancy of a library is generally related to the probability that a specific sequence is actually present within the nucleic acid sequences of that library. Coverage is the ratio of the number of library inserts, such as peptide-encoding oligonucleotides, multiplied by the average insert size divided by the total complexity of the nucleic acid sequences that the library represents.

The term “vector” refers to a nucleic acid sequence that is capable of propagating in a particular host cell and that can accommodate inserts of heterologous nucleic acid. Typically, vectors are manipulated in vitro to insert heterologous nucleic acids into a cloning site. A vector can be introduced into a host cell in a stable or transient manner, such as by transformation or transfection.

The term “expression vector” refers to a vector designed to express an inserted nucleic acid. Such vectors can contain, for example, one or more of the following operably associated elements: a promoter located upstream of the insertion site (e.g., a cloning site) of the nucleic acid, a transcription termination signal, a translation termination signal and/or a polyadenylation signal. An expression vector can also include a selectable marker, such as a drug resistance gene (e.g., hygromycin or neomycin resistance). (See, e.g., Santerre et al., Gene 30:147-56 (1984).)

The term “high copy number” refers to expression on an extracellular surface of a host cell of at least several hundred to several thousand molecules encoded by a library insert.

The term “expression” in the context of a nucleic acid refers to transcription and/or translation of the nucleic acid into mRNA and/or protein.

The term “expression library” refers to a plurality of copies of an expression construct or vector, a majority of the copies of the construct or vector containing inserts of nucleic acid fragments from the genetic library.

The term “presentation molecule” refers to a polypeptide that can be used to display a peptide or polypeptide as part of a fusion protein.

The term “stable expression” refers to the continued presence and expression of a nucleic acid sequence in a host cell for a period of time that is at least as long as that required to carry out the methods according to the present invention. Stable expression can be achieved by integration of the nucleic acid into a host cell chromosome, or engineering the nucleic acid so that it possesses elements that ensure its continued replication and segregation within the host (e.g., an expression vector or an artificial chromosome) or alternatively, the nucleic acid can contain a selectable marker (e.g. a drug resistance gene) so that stable expression of the nucleic acid is ensured by growing the host cells under selection conditions (e.g., drug-containing medium).

The term “specific binding” refers to the direct interaction between a peptide and a target molecule. Such an interaction can be detected either by direct or indirect analysis.

The term “host cell” refers to a cell, particularly a mammalian cell, that can serve as a recipient for a genetic library, and that is introduced by any one of several procedures. The host cell often allows replication and segregation of a vector containing a library insert. In certain embodiments, however, replication and segregation are irrelevant; expression of a library insert is all that is required. Suitable host cells will include normal human cells as well as those from certain disease states, such as neoplasia or cancer. Other suitable cells include Ba/F3, AC2 (see, e.g., Garland and Kinnaird, Lymphokine Res. 5:S145-50 (1986)), B9, HepG2, MES-SA and MES-SA/Dx5 cells. Animal host cells can include, but are not limited to, cells isolated from oncogenic tissues and tumors, including melanocyte, colon, prostate, leukocytes, liver, kidney, uterus, and the like.

The term “phenotype” refers to a measurable characteristic, a change that results from the interaction between peptides and target molecules on the surface of the host cell or that results from the interaction between peptides and target molecules on the surface of any other cell. For example, the measurable characteristic(s) can be associated with the induction or the cessation of apoptosis in target cells, cell survival or growth, receptor-mediated axonal growth and path finding, repair of nerve damage, receptor-mediated cellular differentiation and proliferation, immuno-modulation, leukocyte adhesion-blocking or matrix metalloprotease-inhibition peptides, inhibition or stimulation of autoantibody binding, and the like. The phenotype can also be a measurable binding event between a peptide and any protein or other macromolecular target molecule either attached to a cell or unattached to a cells (e.g., in a complex biological fluid).

The term “reporter” refers to a surrogate for a phenotype. Reporters can be proteins (“reporter proteins”), such as an intracellular or a cell surface protein, that are detectable by antibodies, protein activity (e.g., enzymatic activity), or any other detectable changes.

The terms “target” or “target molecule” refer to a macromolecule (e.g., antibody, soluble protein, cytokine, chemokine, membrane protein, receptor, glycoprotein, proteoglycan, or other macromolecule). A target or target molecule can be localized on the surface of a cell, on the surface of an isolated subcellular organelle, in solution (e.g., a complex biological fluid), or in other extracellular spaces. The terms “extracellular target” or “extracellular target molecule” refer to a macromolecule that is present in an extraceilular space, such as, for example, on the extracellular surface of a cell or in an extracellular biological fluid.

The terms “physiological conditions” and “substantially physiological conditions” refer to conditions that are normally present, or that substantially approximate those normally present, in an extracellular space, on an extracellular surface (e.g. on a cell membrane), and/or in a complex biological fluid. For example, “substantially physiological conditions” can be those present when extracellular targets are active or express their activities (e.g., enzymatic activity, binding to a receptor, substrate, scaffolding molecule, or other binding partner, and the like).

The term “complex biological fluid” refers to a biological fluid, such as, for example, autologous (i.e., from the same animal), homologous (i.e., from an animal of the same species), or heterologous (i.e., from a different species) blood, plasma, serum, a secretion (e.g., sweat, tears, urine, semen, vaginal fluid, or mucous) and the like. Complex biological fluids can be either undiluted or substantially undiluted. The term “substantially undiluted complex biological fluid” refers to a complex biological fluid that is either undiluted or diluted in physiological buffers to typically no less than about 50% concentration. Substantially undiluted complex biological fluids, i.e., no less than approximately 50% of undiluted fluids, have substantially the same ionic composition and strength and substantially the same macromolecular structures in solution, in approximately the same absolute concentrations, as the undiluted fluid.

The terms “transformation” or “transfection” refer to the process of introducing nucleic acids into a recipient (e.g., host) cell. This is typically detected by a change in the phenotype of the recipient cell. The term “transformation” is generally applied to microorganisms, while “transfection” is used to describe this process in cells derived from multicellular organisms.

The term “flow sorter” refers to a device that analyzes light emission intensity from cells or other objects and separates these cells or objects according to parameters such as light emission intensity. Suitable flow sorters include, for example, a fluorescence-activated cell sorter (FACS), a spectrophotometer, microtiter plate reader, a charge coupled device camera and reader, a fluorescence microscope, or similar device.

The terms “bright” and “dim” in the context of a flow sorter refer to the intensity levels of fluorescence (or other modes of light emission) exhibited by particular cells: Bright cells have high intensity emission relative to the bulk population of cells and, by inference, high levels of reporter; dim cells have low intensity emission relative to the bulk population and, by inference, low levels of reporter.

The term “bead selection” refers to the use of beads to selectively remove cells from a mixture of cells. Beads can include a macromolecule, such as an antibody or other binding partner. In certain embodiments, the bead selection uses derivatized magnetic beads. For example, cells expressing a FLAG epitope on the cell surface can be pre-selected on magnetic beads that are coated with anti-FLAG antibody. The magnetic beads can then be collected using a strong magnetic field.

Genetic Libraries

The genetic libraries according to the present invention include a collection of at least partially heterogeneous nucleic acid fragments. Such nucleic acid fragments can include, for example, synthetic DNA or RNA, genomic DNA, cDNA, mRNA, cRNA, heterogeneous RNA, and the like. The nucleic acid fragments can represent, for example, all or some portion of a population of nucleic acids, such as a genome, of a population of mRNAs, or some other set of nucleic acids that contain nucleic acid sequences of interest. The genetic libraries contain sequences in a form that can be manipulated.

The present invention typically uses genetic libraries that are derived from synthetic DNA or from fragments of genomic DNA and/or cDNA from a particular organism. Such library sequences will typically range from about 10 bases to about 10 kilobases. The library sequences can optionally be oligonucleotides having, for example, an average length of from about 10 bases to about 60 bases.

Methods of making synthetic DNA are known to those of skill in the art. (See, e.g., Glick and Pasternak, Molecular Biotechnology: Principals and Applications of Recombinant DNA, ASM Press, Washington, D.C. (1998).) Methods of making randomly sheared genomic DNA and/or cDNA, and of manipulating such DNA's, are also known in the art. (See, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); which are incorporated by reference herein.) The details of library construction, manipulation and maintenance are also known in the art. (See, e.g., Ausubel et al., supra; Sambrook et al., supra.)

In some aspects, the library is made of synthetic nucleic acid fragments. For example, a population of synthetic oligonucleotides representing all possible sequences of length N (where N is a positive integer), or a subset of all possible sequences, can be the nucleic acids for the library. A population of synthetic oligonucleotides encoding all possible amino acid sequences of length N, or a subset of all possible sequences, can also be the nucleic acids for the library. Alternatively, a semi-random library can be used. For example, a semi-random library can be designed according to the codon usage preference of the host cell or to minimize the inclusion of translational stop codons in the encoded amino acid sequence. As an example of the latter, in the first position of each codon, equimolar amounts of C, A, and G and a one half-molar amount of T would be used. In the second position, A is used at a one half-molar amount while C, T, and G would be used in equimolar amounts. In the third position, only equimolar amounts of G and C would be used.

Such synthetic oligonucleotides can optionally include any suitable cis regulatory sequence, such as, for example, a promoter, a translational start codon, a translational termination signal, a transcriptional termination signal, a polyadenylation signal, a cloning site (e.g., a restriction enzyme sites or cohesive end(s)), a sequence encoding an epitope, and/or a priming segment. For example, a library can include DNA fragments having a restriction enzyme site near one end, operably associated with an ATG start codon, a random or semi-random sequence of N nucleotides, a translational stop codon, a primer binding site and a restriction enzyme site at the other end. Such a collection of fragments can be directly ligated into an expression construct, into a vector, into an expression vector, and the like. The fragments can be introduced as single stranded or double stranded DNA, and as either sense or antisense strands. As will be appreciated by the skilled artisan, double stranded nucleic acids can be formed, for example, by annealing complementary single stranded nucleic acids together or by annealing a complementary primer to the nucleic acid and then adding polymerase and nucleotides (e.g., deoxyribonucleotide or ribonucleotide triphosphates) to form double stranded nucleic acids. Double stranded nucleic acids can also be formed by ligating single stranded nucleic acids (e.g., DNA) into a site with 5′ and 3′ overhanging ends and then filling in the partially single stranded nucleic acids with a polymerase and nucleotide triphosphates. The details of manipulating and cloning oligonucleotides are known in the art. (See, e.g., Ausubel et al., supra; Sambrook et al., supra.)

The libraries most typically comprise nucleic acids that have coverage that exceeds the possible permutations of the nucleic acid of the library sequences. For example, a library can comprise a number of nucleic acids that exceeds the possible permutations of nucleic acid sequences by about 5 times, although greater and lesser amounts of redundancy are within the scope of the invention. The details of library construction, manipulation and maintenance are known in the art. (See, e.g., Ausubel et al., supra; Sambrook et al., supra.)

In an exemplary embodiment, a library is created according to the following procedure using methods that are well known in the art. Double stranded DNA fragments are prepared from random or semi-random synthetic oligonucleotides, randomly cleaved genomic DNA and/or randomly cleaved cDNA. These fragments are treated with enzymes, as necessary, to repair their ends and/or to form ends that are compatible with a cloning site in an expression vector. The DNA fragments are then ligated into the cloning site of copies of the expression vector to form an expression library. The expression library is introduced into a suitable host strain, such as an E. coli strain, and clones are selected. The number of individual clones is typically sufficient to achieve reasonable coverage of the possible permutations of the starting material. The clones are combined and grown in mass culture, or in pools, for isolation of the resident vectors and their inserts. This process allows large quantities of the expression library to be obtained in preparation for subsequent procedures described herein.

Expression Cassettes and Vectors

In another aspect, expression cassettes and/or vectors are used to express peptides and/or fusion proteins encoded by sequences of an expression library. There are numerous expression cassettes and vectors known in the art which are readily available for use. (See, e.g., Atisubel et al., supra; Sambrook et al., supra.) Some of these cassettes and vectors are tailored for use in specific cell types, while others can be used in a wide variety of cell types. In mammalian cells, viral transcriptional regulatory elements are a typical choice for driving expression of exogenous coding sequences, such as library sequences. An expression cassette or vector can also include one or more selectable markers to identify host cells that contain the expression vector and/or the expression library.

To effect expression of peptides, an expression cassette can include, for example, in a 5′ to 3′ direction relative to the direction of transcription, a promoter region operably associated with a cloning site for insertion of library sequence and a transcriptional termination region, optionally having a polyadenylation (poly A) sequence. The expression cassette can optionally include a ribosome binding sequence, a translation initiation codon, and/or a translational termination codon. A secretion signal and/or transmembrane domain are typically included adjacent the cloning site.

Suitable secretion signals include, for example, those from CD24. Suitable transmembrane domains include, for example, a signal for glycophosphatidylinositol (GPI) anchorage, the transmembrane domain of CD24, IL-3 receptor, and the like.

To effect expression of the library sequences in host cells of a particular type, a promoter capable of conferring robust, high or moderately high expression of the library insert is preferred. Suitable promoter sequences can include, for example, to an enhancer and/or a TATA box capable of binding an RNA polymerase (such as RNA polymerase II). The promoter can be constitutively active (such as a viral promoters), or it can be inducible. An inducible promoter can be used when controlled expression of library sequences is desired and/or to avoid toxic side affects associated with expression or over-expression of peptide sequences and/or fusion proteins. Suitable inducible promoters include, but are not limited to, interferon inducible promoter systems, the promoters for 3′-5′ poly (A) synthetase or Mx protein (see, e.g., Schumacher et al., Virology 203:14448 (1994)), the HLV-LTR, the metallothionen promoter (see, e.g., Haslinger et al., Proc. Natl. Acad. Sci. USA 82:8572-76 (1985)), the SV40 early promoter region (Bernoist and Chambon, Nature 290:304-10 (1981)), the promoter contained in the 3′ long terminal repeat of Rous sarcoma virus (Yamamoto et al., Cell 22:787-97 (1980)), the herpes thymidine kinase promoter (Wagner et al., Proc. Natl. Acad. Sci. USA 78:1441-45 (1981)), and the like.

Other suitable promoters can be derived from housekeeping genes that are expressed at high or reasonably high levels. For example, the promoter for β-actin is useful for high expression. (See, e.g., Qin et al., J. Exp. Med. 178:355-60 (1993).) Similarly, the cytomegalovirus promoter and the translational elongation factor EF-1α promoter are other strong promoters useful for expression. In general, suitable promoters, such as housekeeping or viral gene promoters, can be identified using well-known molecular genetic methods.

In certain embodiments, the cloning site is adjacent to one or more translational termination sequences, such that the length of any resulting expressed peptide is substantially the same as the coding region of the library sequence. As used herein, the phrase “substantially the same length” means that the length of the expressed peptide corresponds to the length of the coding region in the library sequence and can further encode, for example, a methionine residue corresponding to the start codon, any additional amino acids resulting from linker nucleic acids within the coding region, translational or post-translational modifications, one or more epitopes, and the like.

In some embodiments, the cloning site is flanked by epitopes. Suitable epitopes can include, for example, Xpress® leader peptide (Asp-Leu-Tyr-Asp-Asp-Asp-Asp-Lys, SEQ ID NO:1; InVitrogen), a myc epitope (Glu-Gln-Lys-Leu-Ile-Ser-Glu-Glu-Asp-Leu-Asn, SEQ ID NO:2; InVitrogen); the V5 epitope (Gly-Lys-Pro-Ile-Pro-Asn-Pro-Leu-Leu-Gly-Leu-Asp-Ser-Thr; SEQ ID NO:3), the FLAG tag (Asp-Tyr-Lys-Asp-Asp-Asp-Asp-Lys, SEQ ID NO. 4), see, e.g., Hopp et al., Biotechnology 6:1205-10 (1988)), the lexa protein, thioredoxin, FLAG, polyhistidine, and the like.

In certain embodiments, a cloning site is associated with the coding region of a fusion protein for extracellular display of the peptides (also referred to as a “presentation molecule”). Such a fusion protein can include, for example, (1) homologous protein domains, protein fragments, or proteins as found in the host cell or on the host cell surface, and/or (2) heterologous protein domains, protein fragments, or proteins from another type of cell. The choice of fusion protein depends on the type of host cell(s), the stability of the fusion protein, and the desired conformation of the expressed peptide (e.g., constrained or unconstrained). Such a presentation molecule typically includes a signal sequence and a transmembrane domain.

The presentation molecule can display the peptide at or near the N-terminus, at or near the C-terminus, or internally to the presentation molecule. In an exemplary embodiment, the presentation molecule displays the peptide at the N-terminus and the C-terminal portion of the presentation molecule is anchored to the cell membrane by a transmembrane domain. The presentation molecule can be modified to position the peptides at varying distances from the host cell surface to increase the probability of achieving the appropriate steric orientation for specific binding between peptides and the target (e.g., a receptor).

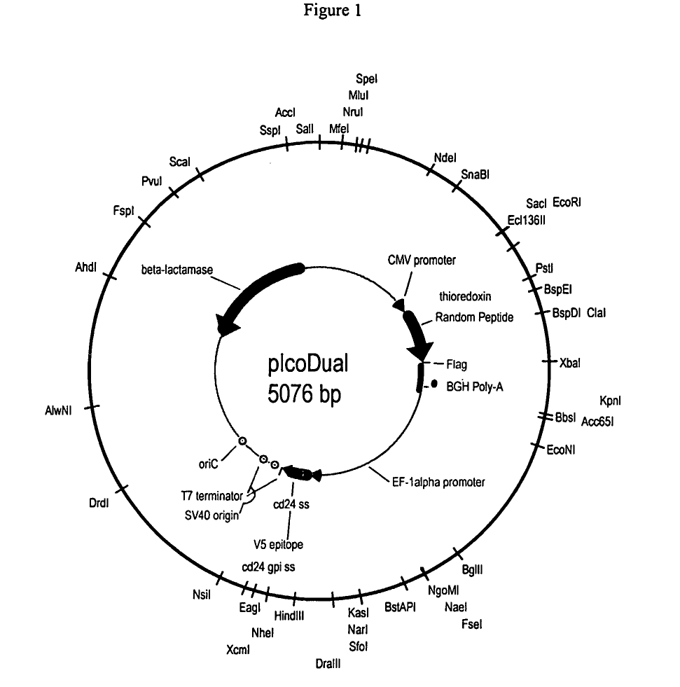

Suitable presentation molecules can include, for example, lymphocyte antigen CD20, modified IL-3 receptor, CD24 (see, e.g., Poncet et al., Acta Neuropathol. (Berl) 91:400-08 (1996)), and the like. Referring to FIG. 1, the pIcoDual vector includes exemplary expression cassettes. For example, one expression cassette encodes a CD24-V5 fusion protein and includes one or more unique restriction sites for insertion of library sequences. Another suitable fusion protein includes E. coli thioredoxin and the FLAG epitope. At the junction between the thioredoxin and FLAG coding sequences, a unique XbaI restriction site permits insertion of library sequences into the fusion protein-coding region.

An expression cassette can optionally be part of an expression vector. Suitable expression vectors are known in the art. (See, e.g., Ausubel et al., supra; Sambrook et al., supra.) In certain embodiments, a controlled plasmid amplification system is used for expression in mammalian cells. Such a system allows controlled plasmid amplification in a variety of cells. Increased plasmid copy number can also lead to increased expression of the encoded peptides. High level expression of the peptides can increase the numbers of peptides displayed on an extracellular surface. Such a controlled amplification system also allows for sustained transient expression in mammalian cells. Sustained transient expression can be advantageous because typically 10 times as many cells exhibit transient expression as compared to stable transfection which can allow larger numbers of peptides to be effectively screened. Plasmid amplification also facilitates recovery of plasmids or sequences encoding peptides of interest.

In an exemplary embodiment, the controlled plasmid amplification system utilizes the SV40 replication system. The expression vector contains a fusion of the early promoter of SV40 and the coding region for Large T antigen, so that transcription of Large T antigen is under the control of the early promoter of SV40. The vector also contains the SV40 origin of replication. When this vector enters a cell, the SV40 early promoter promotes transcription of Large T antigen RNA. The RNA is translated into Large T antigen. Large T antigen binds to the SV40 origin and cause amplification of the plasmid. As Large T antigen concentration rises in the cell, the binding of Large T antigen to the SV40 early promoter shuts down the SV40 early promoter and, consequently, Large T antigen RNA synthesis. The system, therefore, is self-regulating. As the plasmid copy number rises, there will not be an increase in production of Large T antigen that would continue to escalate plasmid amplification to the point of cell death. The amount of Large T antigen in a cell will be a function of the amount of Large T antigen RNA, the stability of the Large T antigen RNA, the stability of Large T antigen protein, the relative affinity for the origin of replication and the SV40 early promoter, and the reduction in the amounts of vector, Large T antigen RNA, and Large T antigen due to cell division. Because the amplification system is contained on a vector, plasmid amplification is typically not limited to the use of COS7 host cells, but rather plasmid amplification can be used for most mammalian cell types.

For other replication systems, the expression vector, if it is of viral origin, may not require propagation in a bacterial host. More typically, however, the vector is propagated in a bacterial host and contains sequences necessary for replication and selection in E. coli, such as, for example, a colE1 replicon and an antibiotic resistance gene.

An expression vector can optionally contain one or more selectable markers. For example, suitable selectable markers for transfection of eukaryotic cells include the genes for hygromycin resistance, neomycin resistance, blasticidin resistance, zeocin resistance, doxorubicin resistance, and the like. Suitable selectable markers for other cells include other antibiotic resistance genes and those complementing auxotrophies (e.g., amino acid auxotrophies). The expression vector can also optionally include a selectable marker to signal that the host cell contains the expression vector. Suitable selectable markers will include green fluorescent protein, or epitopes such as, for example, polyhistidine, the Xpress® leader peptide (Asp-Leu-Tyr-Asp-Asp-Asp-Asp-Lys, SEQ ID NO:1; InVitrogen), a myc epitope (Glu-Gln-Lys-Leu-Ile-Ser-Glu-Glu-Asp-Leu-Asn, SEQ ID NO: 2; InVitrogen; Glu-Gln-Lys-Leu-Ile-Ser-Glu-Glu-Asp-Leu-Asn, SEQ ID NO: 3; InVitrogen), the V5 epitope (Gly-Lys-Pro-Ile-Pro-Asn-Pro-Leu-Leu-Gly-Leu-Asp-Ser-Thr; SEQ ID NO: 4), the FLAG tag (Asp-Tyr-Lys-Asp-Asp-Asp-Asp-Lys, SEQ ID NO: 5), see, e.g., Hopp et al., Biotechnology 6:1205-10 (1988)), the lexA protein, or bacterial thioredoxin. Such markers can be detected, for example, by enzyme assay, by fluorescence using a flow sorter or similar device, using antibodies (e.g., a monoclonal or polyclonal antibody), using bead selection, and the like. When such markers are present on the cell surface, they can be used to isolate or to enrich for cells expressing the marker.

Nucleic Acid Transfer

A variety of methods can be used to transfer library sequences into host cells. (See generally Ausubel et al., supra; Sambrook et al., supra.) Some methods give rise primarily to transient expression in host cells (i.e., the expression is gradually lost from the cell population). Other methods can generate cells that stably express the library sequences, though the percentage of stable expressers is typically lower than transient expressers. Such methods include viral and non-viral mechanisms for nucleic acid transfer.

Suitable mammalian cells include, for example, K562, COS7, Ba/F3, AC2 (see, e.g., Garland and Kinnaird, Lymphokine Res. 5:S145-50 (1986)), B9, HepG2, MES-SA, MES-SA/Dx5 cells, and the like. Animal host cells can include, but are not limited to, cells isolated from oncogenic tissues and tumors, including melanocyte, colon, prostate, leukocytes, liver, kidney, uterus, and the like.

For viral vectors, the library sequences are typically carried into the host cell as part of the viral package. Depending on the type of virus, the nucleic acid can remain as an extrachromosomal element (e.g., adenoviruses (see, e.g., Amalfitano et al., Proc. Natl. Acad. Sci. USA 93:3352-56 (1996)) or adeno-associated virus or it can be incorporated into a host chromosome (e.g., retroviruses (Iida et al., J. Virol. 70:6054-59 (1996)).

For the transfer of non-viral expression vectors, many methods can be used. (See, e.g., Ausubel et al., supra; Sambrook et al., supra.) One method for nucleic acid transfer is calcium phosphate coprecipitation of nucleic acid. This method relies on the ability of nucleic acid to coprecipitate with calcium and phosphate ions into a relatively insoluble calcium phosphate complex, which settles onto the surface of adherent cells on the culture dish bottom. Other methods employ lipophilic cations that bind nucleic acid by charge interactions while forming lipid micelles. These micelles fuse with cell membranes, introducing the nucleic acid into the host cell where it is expressed. Another method of nucleic acid transfer is electroporation, which involves the discharge of voltage from the plates of a capacitor through a buffer containing nucleic and host cells. This process disturbs the cell membrane sufficiently that nucleic acid contained in the buffer is able to penetrate those membranes. Another method involves using cationic polymers, such as DEAE dextran, to mediate nucleic acid entry and expression in cultured cells. Another method employs ballistic delivery of nucleic acid into cells. Finally, microinjection of nucleic acid can be used.

Large numbers of identical vectors (e.g., expression vectors containing library sequences) can be introduced into each mammalian cell by fusing such cells with spheroplasts of bacteria harboring a multi-copy vector. The fusion is performed in a manner that on the average allows for the fusion of one spheroplast with one mammalian cell. For example, when a high copy number plasmid, such as a derivative of a pUC plasmid, is used, many identical plasmids are typically introduced into each mammalian cell. This method circumvents the need for amplification of the vector in mammalian cells, and allows for high copy number in the mammalian cells and the resulting high levels of expression of library sequences. This procedure can also provide for longer periods of transient expression without a need to amplify the vectors in mammalian cells.

High copy numbers of vector also increase the ease with which library sequences can be recovered from mammalian cells which exhibit a change in reporter expression.

In some of these methods, multiple nucleic acids which can encode polypeptides which might interact with a target molecule are introduced into individual cells. Methods are known in the art to minimize transfer of multiple fragments. For example, by using “carrier” nucleic acid (e.g., DNA such as salmon or herring sperm DNA, tRNA, and the like), or by reducing the total amount of nucleic acid applied to the host cells, the problem of multiple fragment entry can be reduced. In addition, each recipient cell can receive multiple nucleic acid fragments. Multiple passages of the library through the host cells permit sequences of interest to be separated ultimately from other sequences that can be present initially as false positives.

Detection of Peptide Binding to Extracellular Target Molecules

In another aspect, the interaction of peptides in the libraries with target molecules is assayed. Screening is typically conducted under physiological conditions to identify peptides that interact with target molecules. Examples of suitable target molecules include, but are not limited to, tumor-specific antigens (TSA); tumor-associated antigens (TAA); extracellular surface macromolecules; cell surface receptors; cytokines, chemokines, or other factors that bind to cell surface receptors; antibodies, such as autoantibodies associated with disease; soluble proteins in complex biological fluids, such as enzymes in the blood cascade pathways; and the like. The target molecules can be present on the extracellular surface of mammalian cells, animal cells, viruses, parasites, and the like.

The peptides are typically displayed under substantially physiological conditions on the surface of host cells, such as mammalian cells. Each host cell can express on its surface hundreds and possibly thousands of copies of one or more library peptides, a majority of which are typically available for binding to extracellular target molecules. The peptides are typically present on the surface of a cell for a sustained period of time.

In an exemplary embodiment, contacting of displayed peptides with target molecules is performed under substantially physiological conditions, such as in the presence of a complex biological fluid. For example, soluble target molecules in a complex biological fluid can be contacted with host cells, such as mammalian cells, displaying the peptides on an extracellular surface to form complexes. The peptides of the library can be contacted with target molecules, such as cytokines, chemokines, autoantibodies, or other soluble factors present in blood.

In certain embodiments, the target molecules can be labeled. Suitable labels include, for example, radioactive labels (e.g., ³H, ¹⁴C, ³²P, ³⁵S, ¹²⁵I, ¹³¹I, and the like), fluorescent molecules (e.g., fluorescein isothiocyanate (FITC), rhodamine, phycoerythrin (PE), phycocyanin, allophycocyanin, ortho-phthaldehyde, fluorescamine, peridinin-chlorophyll a (PerCP), Cy3 (indocarbocyanine), Cy5 (indodicarbocyanine), lanthanide phosphors, and the like), enzymes (e.g., horseradish peroxidase, β-galactosidase, luciferase, alkaline phosphatase), biotinyl groups, epitopes (supra), and the like. In some embodiments, detectable labels are attached by spacer arms of various lengths to reduce potential steric hindrance. Alternatively, labeled binding partners, such as, for example, antibodies that bind to the target molecules can be used.

In certain other embodiments, the host cell and/or target cells (i.e., cells having a target molecule on an extracellular surface) can be labeled. Suitable labels can include, for example, PKH26, that inserts into cell membranes in a stable non-toxic manner (see, e.g., Horan et al., Methods Cell Biol. 33:469-90 (1990)). Other labels can be, for example, cell surface markers, other molecules present on the cell surface, or proteins or other molecules within a cell. Labels within a cell can include, for example, β-galactosidase (Shapiro et al., Gene 25:71-82 (1983)), the CAT gene from bacteria (Thiel et al., Gene 168:173-76 (1996)), the luciferase gene from firefly (Gould and Subramani, Anal. Biochem. 175:5-13 (1988)), fluorescent markers such as green fluorescent protein (GFP) and derivatives thereof (see, e.g., U.S. Pat. No. 5,491,084), or other detectable macromolecules within a host cell and/or target cell. Target or host cells can be detected using labeled binding partners, such as, for example, anti-CD71 or anti-CD24 antibody labeled with biotin, and the like. In certain other embodiments, a reporter on a target cell or a host cell can signal binding of a peptide library-expressing cell to the target cell. Such a reporter is a detectable cellular component, such as proteins, enzymes, heterologous proteins or enzymes, or other macromolecules.

In certain embodiments, the host cells expressing the peptide libraries are freshly prepared or live cells. In other embodiments, the peptide library expressing cells can be fixed, such as in para-formaldehyde or other suitable fixative. Such fixed peptide library-expressing cells optionally can be stored at a suitable temperature (e.g., 4° C.) until use. The peptides are typically presented on the surface of the cells for sustained period of times. In some embodiments, the host cells expressing the peptides can remain bound to target molecules or cells for extended periods of time without removal by endocytosis or other cellular processes.

Following contacting of the target molecules with the displayed peptide libraries, washing can optionally be performed to separate non-specifically bound target molecules from peptides. Suitable washing buffers will include, for example, unlabeled complex biological fluids, substantially undiluted complex biological fluids, and the like.

Interaction of peptides in the libraries with the target molecules can be detected by any suitable detection means, such as, for example, florescence activated cell sorting (FACS) analysis, bead selection, magnetic bead selection, and the like. For target molecules that have enzymatic activity, target molecules bound to library peptides can be detected by enzyme activity assay. In certain embodiments, target molecule-peptide interaction can be detected by the presence or absence of enzymatic activity. Suitable enzymatic assays include calorimetric, fluorescent, and the like. Such assays can be performed, for example, in a microtiter plate, in tissue culture plates, and the like.

In another exemplary embodiment, library peptides can be identified that specifically interact with tumor-specific antigens (TSA) or tumor-associated antigens (TAA) on cells, such as cancer cells. The library peptides can be expressed on any suitable cell type, such as mammalian cells (e.g., K562 cells). For adherent target cells, host cells containing the peptide library are typically contacted with the target cells in tissue culture plates or other adherent medium under physiological conditions.

In one example, cancer cells are provided adhered to tissue culture plates. Peptide library-expressing K562 cells are placed in the plates and allowed to contact the target cells under physiological conditions. The K562 cells can settle onto the cancer cells and, after an appropriate incubation time, unbound cells are removed during several rinse procedures typically involving aspiration of medium containing unbound cells, followed by addition of fresh medium, gentle agitation to suspend unbound cells, and aspiration of the medium containing unbound cells. The rinse procedure optionally can be repeated at least two additional times. K562 cells are non-adherent and, therefore, any K562 cells that remain attached to the target cells are those expressing library peptides that interact with surface molecules on the cancer cells. The bound peptide library-expressing K562 cells can then be removed and collected using an epitope (e.g., a FLAG epitope co-expressed with the peptide libraries) or other suitable label on marker or associated with the peptide libraries. Such collection can be performed, for example, by bead selection, magnetic bead selection, FACS analysis, and the like.

Cancer cells can have higher numbers of TAA and/or TSA on the cell surface as compared with non-cancerous cells. Therefore, relatively fewer target cells (e.g., a few million cells) can provide a large number of potential TSA or TAA molecules to interact with the peptide libraries on the host cells. For example, 2 million to 5 million adherent target cells can be accommodated without crowding in a plastic tissue culture plate having a surface area of 150 cm². Cells expressing a peptide library according to the present invention can be screened in a single plate containing up to 5 million target cells and large numbers (e.g., millions or hundreds of millions) of peptide library-expressing cells under physiological conditions. Occasional, gentle agitation of the plate can optionally ensure that the peptide library-expressing host cells are exposed to the target cells during contacting (e.g., an about 30 to about 60 minute incubation period). Following removal of the non-binding peptide library-expressing host cells, the host cells and target cells can be collected (e.g., lifted) from the plate. The collected cells can be applied to FACS analysis or bead selection. The collected cells (typically only about a few to about 5 million cells, or less) are recovered. Additional rounds of selection can be performed to enrich for host cells expressing specific peptides. Additionally, contacting of the collected sub-library with non-cancerous (e.g., normal cells) can be used to determine whether the peptides in the sub-library interact with a normal complement of TSA and/or TAA on non-cancerous cells. Alternatively, the peptide library-expressing cells can be pre-adsorbed onto cultures of normal cells (e.g., fibroblasts or epithelial cells) and the recovered, non-adherent, peptide library-expressing cells then used to screen target cells (e.g., cancerous cells).

The target cells can be also non-adherent cells, such as, for example, non-adherent cancer cells (e.g., leukemia cells). Interactions between non-adherent target cells and peptides in the library can be detected by stable complex formation between host cells expressing the peptide library and the target cells. Stable complex formation can occur, for example, when high avidity library peptides bind to molecules on the surface of the target cells. Following contacting and complex formation, a variety of techniques are available to detect the complexes. For example, many human leukemia cells express abundant levels of CD71 (transferrin receptor), CD24, or other common surface markers to which specific antibodies (e.g., monoclonal or polyclonal antibodies) are available. Other suitable methods include FACS analysis, bead selection, magnetic bead selection, and the like.

In one example, a convenient screen for TSA or TAA on non-adherent leukemia cells can be conducted by first tagging the target cells with anti-CD71 or anti-CD24 antibody labeled with biotin. Small (e.g., 50 nanometer) magnetic beads labeled with streptavidin can then be introduced to coat the cells. The cells are captured in a magnetic field on a column (e.g., Miltenyi Biotech, Germany) that allows cells without magnetic beads to pass through the column while cells bound to magnetic beads are retained in the column. The target cells can be loaded onto the column in a manner that results in the uniform distribution of the cells in the column. While the magnetic field is in place, the cells remain attached to the column. Peptide library-expressing host cells can be introduced into the column typically under physiological or substantially physiological conditions. Peptide library expressing host cells that bind with high avidity to target leukemia cells are retained in the column during washing procedures. The magnetic field then is removed, and the cells in the column are recovered. Subsequent rounds of selection can be performed to enrich for cells expressing high avidity library peptides.

In another exemplary embodiment, FACS analysis is used to identify library peptides that bind to TAA and/or TSA on non-adherent cells. Two different labels can be used to detect complexes between host cells expressing a peptide library and the target cells. One label is on the target cells, and the other label is on the peptide library-expressing host cells. For example, target leukemia or other non-adherent target cells can be tagged with anti-CD71 or anti-CD24 antibody labeled with biotin, FITC, an aliphatic fluorescent dye, PKH26, that inserts into cell membranes in a stable non-toxic manner (see, e.g., Horan et al., Methods Cell Biol. 33:469-90 (1990)), or other suitable label. Peptide library-expressing cells (e.g., K562 cells) have a second, different label, such as a FLAG epitope, an aliphatic fluorescent dye, PKH26, or other suitable label. The peptide library-expressing cells then can be contacted with the target cells and complex formation allowed to occur. In one procedure, anti-FLAG antibody labeled with a non FITC-like fluorophore can be added to label the peptide library-expressing cells. During FACS analysis, cell complexes containing both labels are collected, while uncomplexed cells are not collected. Alternatively, the target cells can be tagged with a biotin-labeled antibody and subsequently collected on magnetic beads prior to FACS analysis to detect complexes of target cells and peptide library-expressing host cells. This procedure can be used to remove large numbers (e.g., hundreds or millions) of unbound peptide library-expressing cells prior to FACS analysis.

In other embodiments, the methods according to the present invention can be used to identify library peptides that bind to surface molecules on bacteria, fungi, viruses, parasites, or isolated subcellular organelles under physiological conditions. For example, bacteria, fungi, viruses, and parasites can be screened with host cells expressing a peptide library according to the present invention under physiological conditions for peptides that bind to a surface molecule. In another example, library peptides can be identified that bind to bacteria, fungi, viruses, parasites, and the like, in the presence of a complex biological fluid (e.g., blood). Complexes between host cells expressing the library peptides and the target molecule (e.g., on bacteria, fungi, viruses, parasites, and the like) can be detected using any suitable labels and/or techniques, as discussed herein.

Host cells expressing a peptide library that are isolated or collected by any of the methods described herein can be used to re-isolate the genetic library sequences(s) so as to build a sub-library of sequences enriched for those that bind to the target molecule(s). As will be appreciated by those skilled in the art, such sequences can be isolated by, among other methods, recovering expression vector nucleic acids from the selected clones and transforming them into a suitable bacterial host strain, by cloning the library oligonucleotides by PCR using any suitable priming site(s) that flanks the oligonucleotide inserts, by subcloning the oligonucleotides from the original expression vector into another vector, and the like. The sub-library of oligonucleotides optionally can be recloned into an expression cassette or vector, as necessary, and reintroduced into the host cells for subsequent rounds of screening. This screening/selection cycle can be repeated as many times as necessary.

In certain embodiments, after a sufficient number of cycles, a substantial difference is observed in binding between an enriched sub-library of peptide sequences and the original peptide library (e.g., in an intensity distribution). By the process of sequential introduction of the expression library, or portions thereof, sorting in a flow sorter or similar device and isolation of nucleic acids from host cells exhibiting the desired binding to the target molecules, a population of library oligonucleotides can be identified that encode the desired peptide(s). The oligonucleotides can then be isolated and studied individually by molecular cloning and nucleic acid sequence analysis. If a sufficient number of cycles has been carried out, many, and typically most, separate oligonucleotides should encode a peptide that produces about the same effect-binding event when expressed on host cells.

Characterization of Library Sequences

Library sequences isolated using any of the procedures described herein can be further characterized. Library sequences can be isolated from host cells by any suitable method, such as, for example, HIRT lysis and recovery of vectors in bacterial host cells, polymerase chain reaction, and the like. (See, e.g., Hirt, J. Mol. Biol. 26:365-69 (1967); U.S. Pat. Nos. 4,683,202, 4,683,195 and 4,800,159; Innis et al., PCR Protocols: A Guide to Methods and Applications, Academic Press, Inc., San Diego, Calif. (1989); Innis et al., PCR Applications: Protocolsfor Functional Genomics, Academic Press, Inc., San Diego, Calif. (1999); White (ed.), PCR Cloning Protocols: From Molecular Cloning to Genetic Engineering, Humana Press, (1996); EP 320 308; Ausubel et al., supra; Sambrook et al., supra; which are incorporated by reference herein in their entirety.)

Subsequent rounds of screening optionally can be performed to enrich for peptides that interact with the target molecules. Sub-libraries of library peptide sequences can be passed through additional screens and/or selections to enrich for those sequences that have more desirable properties. To enrich for library peptide sequences that have more favored properties, it can be desirable to passage a sub-library (that has been isolated by any of the methods described above) through additional screens to enrich for those peptides with improved specificity, avidity, and the like. For instance, minor effects on an extracellular interaction can be eliminated by appropriate secondary screens. If desired, additional labels can be used to identify library peptide sequences that affect extracellular binding events. In addition, library peptide sequences that have generalized, non-specific effects on extracellular binding events can be identified by passing sub-libraries or individual library peptide sequences through first host cells, or through second, different host cells, and then conducting screens on those cells with different target molecules or with different cell types expressing different target molecules.

In some cases, peptides identified according to the present invention can be used to identify further peptides that interact the target molecules. For example, in some cases, an original library may not contain all possible permutations of an amino acid sequence of length N (e.g., when the original library is a semi-random library). In such cases, it can be possible to isolate and use the identified peptides as a starting material (“lead compound”) to identify additional peptides or peptides with enhanced function (e.g., higher avidity or affinity) as compared with the original peptide. To isolate variants of a library sequences, amplification of nucleic acids (e.g. by polymerase chain reaction) can be used to introduce sequence changes during the replication process. (See, e.g., Cline et al., Nucleic Acids Res. 24:3546-51 (1996).) Such mutations can lead to sequence variants that have more effective properties. Alternatively, it can be desirable to seek improved variants of existing sequences by deliberately subjecting the amplification process to conditions that enhance mutation and/or recombination of the nucleic acid(s), such as by, for example, in vitro mutagenesis, error-prone PCR and/or recombinational PCR. (See, e.g., Ausubel et al., supra; Stemmer, Nature 370:389-91 (1994).) Such conditions are known in the art and provide a means for searching for sequences that are active at lower concentrations and/or that demonstrate increased specificity and/or activity compared to the sequences expressed by the original library.

Applications of the Displayed Peptide Libraries

The methods according to the present invention provide the ability to identify physiologically relevant peptides that bind a wide variety of extracellular target molecules under physiological conditions. Through the combination of extracellular surface-expressed peptide libraries and screening assays, methods according to the present invention can provide tools to identify peptides that bind to known or unknown target molecules. The extracellular target molecules can be a wide variety of molecules. Extracellular cell surface receptors can be identified by binding of a peptide with the receptor. Peptides can also be identified that bind to secreted proteins, such as a soluble protein or a component of the extracellular matrix. Similarly, peptides can be identified that bind to tumor-specific antigens (TSA); tumor-associated antigens (TAA); extracellular surface macromolecules; cytokines, chemokines, or other factors that bind to cell surface receptors; antibodies, such as autoantibodies expressed in disease; and the like. In certain embodiments, the screens can be conducted in the presence of complex biological fluids, such as undiluted blood, plasma, or serum. Such screens facilitate the identification of peptides that bind to target molecules under complex physiological conditions. Because such peptides can act through interaction with extracellular target molecules, the peptides will not be required to enter cells to be effective, will be easier to delivery, and may require lower therapeutic dosages.

In certain embodiments, the identified peptides can be used to alter cellular functions. For example, binding of a peptide to a secreted target protein can alter a target's interaction with a cell surface receptor or another binding partner. Similarly, a peptide can alter function of an extracellular enzyme, such as, for example, a matrix metalloprotease or other component of a protein degradation pathway, by binding to the active site or an allosteric regulatory site of that enzyme. Also, a peptide can inhibit the cell surface receptor's interaction with a ligand, including, for example, a counter-receptor expressed on the surface of the same or another cell type, by binding to the receptor or its ligand.

In various embodiments, peptides can be identified that are specific activators or inhibitors of cell surface receptors, such as cytokine receptors, chemokine receptors, and the like. For example, novel peptide activators of the thrombopoietin (TPO) receptor can be identified using a TPO-dependent cell line as a target cell. Such a cell line can be screened for peptides that can sustain factor-independent growth. Peptides can also be identified that up regulate or down regulate Th1 and/or Th2 immune responses by, for example, screening for peptides that induce or block Th1 or Th2 differentiation through the IL-12 or IL-4 cytokine receptors, respectively. Peptide activators or inhibitors of co-stimulatory pathways in lymphocytes can also be identified by, for example, either screening for peptides that, in conjunction with stimulation through specific receptors (e.g., the T-cell receptor or surface IgM), synergistically promote proliferation of T or B lymphocytes or, alternatively, by screening for peptides that bind specific co-stimulatory receptors (e.g., CD28 or CD40) expressed on T or B lymphocytes followed by functional assays. Similarly, the methods according to the present invention can be used to identify peptides that increase or decrease apoptosis (i.e., programmed cell death) in cells via specific interaction with surface receptors that influence apoptosis. Such receptors can be apoptosis-inducing proteins (e.g., Fas), survival-promoting receptors (e.g., nerve growth factor), or can affect apoptosis-associated functions.

In additional embodiments, by identifying peptides according to their ability to bind specific antibodies, the methods can be used for epitope mapping. Conventional peptide screening methods have been used to determine the epitopes or antigen binding structures of antigenic targets that interact with specific antibody (see, e.g., McConnell et al., Gene 30:115-18 (1994); Harlow and Lane (eds), Using Antibodies: A Laboratory Manual, Cold Spring Harbor Laboratory Press, Cold Spring Harbor, N.Y. (1999)). This approach has several applications including, for example, the determination of the surface topography of antibodies and proteins for which 3-dimensional structures are unknown, the identification of potential inhibitors of antibody-antigen complex formation, and the use of the amino acid sequences of specific peptides in the search of unknown antigens.

Similarly, the methods according to the present invention can be used to identify peptides that specifically bind to antigenic binding sites of autoantibodies that are produced in many diseases. Such peptides can be competitive inhibitors of the natural antigens of autoantibodies. Alternatively, the autoantibody-specific peptides can be used for diagnostic tests. Elevated levels of circulating autoantibodies can correlate with the severity of the particular disease and, therefore, regular measurement of antibody levels can aid in disease management. Peptides when used in diagnostic tests can offer stable, low cost alternatives to natural protein antigens. Also, the methods can be used to determine the specificity of autoantibodies that have unknown antigenic targets. Typically, such antibodies can be obtained from patients by adsorption of circulating antibodies to cells and tissues involved in a particular disease.

Further, methods according to the present invention can be used to identify peptides that specifically bind to soluble proteins or other soluble macromolecular target molecules. Unlike conventional methods, in which the targets are immobilized to a non-physiological surface, screening can be conducted with the target molecules under physiological conditions, such as in blood, plasma, or serum, a physiological environment in which soluble proteins or macromolecules expresses their activity. As used in the present invention, target molecules can be freely accessible to interact with peptides as the targets would when they express activities. Additionally, in certain embodiments the peptides, placed at the N-terminus of an extended presentation molecule, are presented to target molecules relatively distant from the host cell surface and in a favorable hydrophilic environment. Soluble target molecules can include, for example, cytokines (e.g., interleukins), chemokines, any ligand interacting with a cell receptor, hormones, peptide hormones, enzymes such as those in the blood cascade pathways, inhibitors of enzymes, transport proteins such transferrin, proteins associated with drug binding, effector domains of antibodies (e.g., Fc domains), anti-microbial toxins or statins, substances secreted by nicroorganisms or parasites, and the like.

In other applications, peptides that affect normal cellular or biological processes through interaction with extracellular target molecules can be identified. Such peptides can include, for example, those that affect growth rate, morphology, height, weight, fat deposition, and the like. Individual peptides also can be examined in further detail using assays other than the assay used for their isolation. The mechanistic basis for peptide activity can be useful in elucidating biological, genetic and/or biochemical event.

In some embodiments, multiple peptides can be identified that induce or alter an extracellular binding event. Using further rounds of selection, it can be possible to place the peptides into groups based on their effect(s) on the particular binding event(s) or resulting cellular phenotype. For example, the peptides can be grouped based on their effect on apoptotic, nerve growth or function, immuno-modulation, inflammation, and the like. For diseases with multiple phenotypes, it can be possible to examine the effect of the peptides on each phenotype, and to classify those peptides according to their effect on each phenotype.

Additional Modifications to Enhance Peptide Function

As discussed previously, peptide sequences that affect extracellular binding events can exert their effect in a variety of ways. As will be appreciated by those skilled in the art, it can be possible to improve the effectiveness of a peptide by synthesizing peptide variants or analogs. For example, the effectiveness of peptides might be improved by administering the peptides themselves (i.e., without any extra sequences). A wide variety of procedures exist for synthesis of peptides, such as solid-phase synthesis. For example, an amino acid can be bound to a resin particle, and the peptide generated in a stepwise manner by successive additions of protected amino acids to produce a chain of amino acids. Modifications of this technique are commonly used. (See, e.g., Merrifield, J. Am. Chem. Soc. 96:2989-93 (1964); Stewart and Young, Solid Phase Peptide Synthesis, 2nd ed., Pierce Chemical Company, Rockford, Ill. (1984).) In an example of an automated solid-phase method, peptides are synthesized by loading the carboxy-terminal amino acid onto an organic linker (e.g., PAM, 4-oxymethyl phenylacetamidomethyl, and the like), which is covalently attached to an insoluble polystyrene resin cross-linked with divinyl benzene. The terminal amine can be protected by blocking with t-butyloxycarbonyl. Hydroxyl- and carboxyl-groups are commonly protected by blocking with O-benzyl groups. Synthesis can be accomplished in an automated peptide synthesizer, such as that available from Applied Biosystems (e.g., Model 430-A, Applied Biosystems, Foster City, Calif.).

Following synthesis, the product is removed from the resin. The blocking groups are removed, for example, by using hydrofluoric acid or trifluoromethyl sulfonic acid according to established methods. (See, e.g., Bergot et al., Applied Biosystems Bulletin (1987).) A routine synthesis can produce 0.5 mmole of peptide-resin. Following cleavage and purification, a yield of approximately 60 to 70% is typically produced. Purification of the peptides can be accomplished by, for example, crystallizing the peptide from an organic solvent, such as methyl-butyl ether, then dissolving in distilled water and using dialysis (if the molecular weight of the subject peptide is greater than about 500 daltons) or reverse high-pressure liquid chromatography (e.g., using a C18 column with 0.1% trifluoroacetic acid and acetonitrile as solvents) if the molecular weight of the peptide is less than 500 daltons. Purified peptide can be lyophilized and stored in a dry state until use.

One skilled in the art will appreciate that structural analogs and derivatives of peptides (e.g., peptides having conservative amino acid insertions, deletions or substitutions, peptidomimetics, and the like) can also be useful as therapeutic agents. For example, in addition to the above-described peptides, which can comprise naturally occurring amino acids, peptide analogs can be used as non-peptide drugs with properties analogous to those of the template peptide. These types of non-peptide compounds can be developed, for example, with the aid of computerized molecular modeling. (See, e.g., Fauchere, Adv. Drug Res. 15:29 (1986); Veber and Freidinger, TINS 392 (1985); Evans et al., J. Med. Chem. 30:1229 (1987).) Such analogs, or peptide mimetics, are structurally similar to therapeutically or prophylactically useful peptides and can be used to produce an equivalent therapeutic or prophylactic effect. In some cases, peptide mimetics can have significant advantages over peptides, including, for example, more economical production, greater chemical stability, enhanced pharmacological properties (e.g., half-life, absorption, potency, efficacy, and the like), altered specificity (e.g., a broad-spectrum of biological activities), increased or reduced antigenicity, and others.

Peptide mimetics can be generated by methods known in the art and further described in the following references: Spatola, Chemistry and Biochemistry of Amino Acids, Peptides, and Proteins (Weinstein, ed.) 267 (1983); Spatola, Vega Data, Vol. 1, Issue 3, “Peptide Backbone Modifications” (March 1983); Morley, Trends Pharm Sci., pp. 463-68 (1980); Hudson et al., Int. J. Pept. Prot. Res. 14:177-85 (1979); Spatola et al., Life Sci. 38:1243-49 (1986); Hann, J. Chem. Soc. Perkin Trans. I, pp. 307-314 (1982); Alnquist et al., J. Med. Chem. 23: 1392-98 (1980); Jennings-White et al., Tetrahedron Lett. 23:2533 (1982); European Patent Application EP 45665 (1982); Chemical Abstract 97:39405 (1982); Holladay et al., Tetrahedron Lett. 24:4401-04 (1983); and Hruby, Life Sci. 31:189-99 (1982).

In one aspect, pharmaceutically acceptable salts of a peptide (or analog or mimetic) can be readily prepared by conventional methods. For example, such a salt can be prepared by treating the peptide with an aqueous solution of the desired pharmaceutically acceptable metallic hydroxide or other metallic base and then evaporating the resulting solution to dryness, typically under reduced pressure in a nitrogen atmosphere. Alternatively, a solution of a peptide can be mixed with an alkoxide of the desired metal, and the solution subsequently evaporated to dryness. The pharmaceutically acceptable hydroxides, bases, and alkoxides encompass those with cations for this purpose, including, but not limited to, potassium, sodium, ammonium, calcium, and magnesium. Other representative pharmaceutically acceptable salts include hydrochloride, hydrobromide, sulfate, bisulfate, acetate, oxalate, valerate, oleate, laurate, borate, benzoate, lactate, phosphate, tosylate, citrate, maleate, fumarate, succinate, tartrate, and the like.

It can be desirable to stabilize the peptides or their analogs or derivatives to increase their shelf life and pharmacokinetic half-life. Shelf-life stability can be improved by adding excipients such as: a) hydrophobic agents (e.g., glycerol); b) sugars (e.g., sucrose, mannose, sorbitol, rhamnose, or xylose); c) complex carbohydrates (e.g., lactose); and/or d) bacteriostatic agents. The pharmacokinetic half-life of the peptides can be modified by coupling to carrier peptides, polypeptides, and carbohydrates using chemical derivatization (e.g., by coupling side chain or N- or C-terminal residues), or by chemically altering an amino acid of the subject peptide. The pharmacokinetic half-life and pharmacodynamics of these peptides can also be modified by: a) encapsulation (e.g., in liposomes); b) controlling the degree of hydration (e.g., by controlling the extent and type of glycosylation of the peptide); and c) controlling the electrostatic charge and hydrophobicity of the peptide.

### EXAMPLES

The following examples are provided merely as illustrative of various aspects of the invention and should not be construed to limit the invention in any way.

### Example 1

### Preparation of a Genetic Library in a Host Cell

A genetic library is prepared by inserting random oligonucleotides into a cloning site of an expression vector. The expression vector has an expression cassette comprising, in a 5′ direction relative to the direction of transcription, a promoter, a nucleic acid encoding a signal sequence, a nucleic acid encoding a presentation molecule, a cloning site located at the 5′ end of the nucleic acid encoding the presentation molecule, a nucleic acid encoding a transmembrane domain, and a transcription terminator. The expression vector include an origin of replication (ColEI) and an antibiotic resistance marker for selection in E. coli. The random oligonucleotides encode peptides of about 12 to about 20 amino acid residues. The vectors containing the oligonucleotides are transformed into host bacteria and grown under selectable conditions to establish a library of 10 million to several billion independent isolates. Vector DNA is prepared from this library. This vector DNA is used to transfect animal cells, such as, for example, human cells, mammalian cells or other animal cells.

### Example 2

### Expression Vector Construction

To achieve high expression levels of library peptides on the surface of host cells, a transient expression vector is used. The transient expression vector includes markers required for propagation and selection in bacteria, an expression cassette including a mammalian cell transcription promoter (e.g., the cytomegalovirus or EF-1α promoter), a nucleic acid encoding a presentation molecule and a transcription terminator. Random library sequences can be inserted at the N-terminus, at the C-terminus, or internally in the nucleic acid encoding the presentation molecule and can be in a linear or constrained loop array or in exposed loop of the presentation molecule.

To attach the presentation molecule to the surface of host cells, the nucleic acid encoding the presentation molecule includes a sequence encoding a secretory signal sequence and an element to tether the fusion protein to the cell surface (e.g., a signal for glycophosphatidylinositol (GPI) anchorage or a transmembrane and intracellular domain). Suitable presentation molecules include, for example, the IL-3 receptor, thioredoxin, CD4, CD20, or CD24. The presentation molecule can also include one or more epitopes, such as, for example, FLAG, V5 or polyhistidine. The transcription terminator, can be, for example, from human growth hormone.

### Example 3

### Expression Vector Construction

Two distinct expression vectors were constructed to display peptide libraries on the surface of cells of mammalian cells such as COS7 and K562 cells. One construct places the peptides, having 12 amino acid residues, at the N-terminus as a linear structure, while the other construct includes a cysteine residue at each end of the peptide sequences to form a constrained loop at the N-terminus. Each construct encodes a presentation molecule including thioredoxin, the V5 and FLAG epitopes, the secretory signal sequence from CD24 for secretion, and the GPI linkage sequence from CD24 for attachment to the surface of the host cell. The approximate diversity of each of the completed peptide expression vectors is about 1×10⁹ unique peptides, although libraries of considerably greater diversity can be produced.

### Example 4

### Enrichment of Transfected Cells

Cells vary in the efficiency of transfection. In cases where transfection is not very efficient, it can optionally be possible to enrich for cells that contain the expression vector (e.g., plasmid), and then screen those cells for binding to a target molecule. To allow enrichment of cells that contain the genetic library, a label, such as a sequence encoding a marker, is included in the expression vector. Such a marker typically will remain attached to the cell. This sequence encoding the marker can be, for example, a transcription promoter and terminator, a sequence encoding a secretory signal sequence (e.g., CD24), one or more extracellular domains (e.g., V5, FLAG, and the like) and a sequence to tether the marker to the cell surface (e.g., a signal for glycophosphatidylinositol (GPI) anchorage). The placement of peptide libraries between two distinct markers (e.g., V5 and FLAG) can ensure the integrity of the peptide library by selecting only cells that contain both markers in the presentation molecule and that are expressed on the surface of host cells.

The expression vectors described in Example 3 were used to transfect COS7 cells by electroporation employing conditions to introduce, on average, only one or a few vectors per cell. The cells were placed in culture following transfection and analyzed at various times by FACS to detect the expression of thioredoxin or FLAG on the surface or interior of cells. In each case, a relatively high percentage of cells express the presentation molecules (e.g., thioredoxin—detected using anti-thioredoxin antibody) one day post-transfection, and the molecules persisted over the next week. These results indicate that presentation molecules are expressed in stable form at relatively high levels over several days.

Transfected host cells, expressing a peptide library, were also fixed in para-formaldehyde prior to FACS analysis or selection on magnetic beads. The results demonstrated that the marker epitopes were still accessible to antibody following fixation, indicating that the library peptides were available for binding to target molecules.

Similar experiments demonstrated that K562 cells, transfected by electroporation with plasmid vectors encoding a peptide library, were transfected at approximately 50% efficiency with 80% cell survival. The optimum expression period was between one and two days following transfection. The level of expressed presentation molecule on K562 cells was lower than that on COS7 cells, because K562 cells do not amplify the plasmid vectors as do COS7 cells. However, sufficient presentation molecules were expressed on the surface of K562 cells as demonstrated by localization of labeled anti-FLAG antibody.

The results demonstrate a robust system for displaying peptide libraries on the surface of mammalian cells. The tethering of the presentation molecule via a GPI linkage to the cell and the use of domains from thioredoxin and CD24 lead to the persistent, stable expression of peptide libraries. Further, the placement of the peptides at the N-terminus of the presentation molecule ensures unobstructed accessibility of the peptides to potential target molecules, relatively distant from the cell surface and in a highly favorable hydrophilic environment.

### Example 5

### Identification of Peptides that are Cleaved by Proteases and Other Peptides that Inhibit Proteases

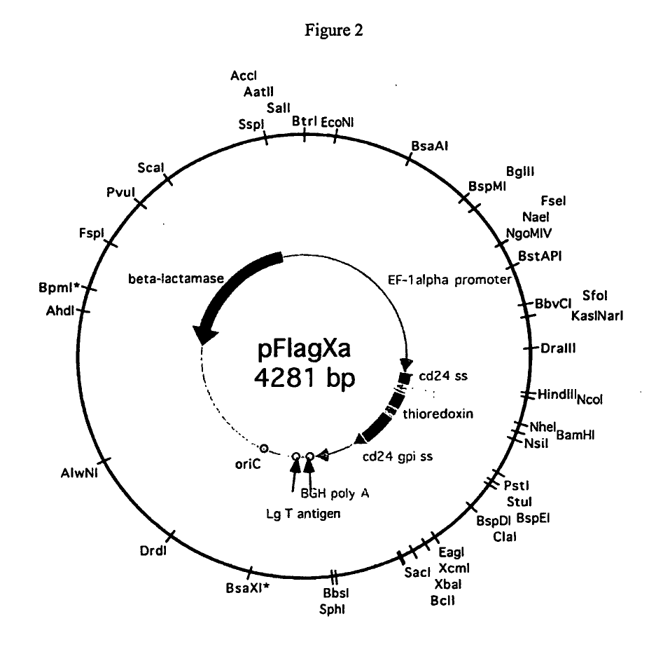

The results of studies completed provided evidence that extracellular displayed peptides could be cleaved by proteases under physiologic conditions. Two plasmids were constructed and expressed in COS7 cells to display fusion proteins on the cell surface anchored via GPI. The protein product of one plasmid, designated pIcoFLAGXa (FIG. 2), consisted of an amino terminal FLAG epitope, followed by the Factor Xa cleavage sequence Ile-Glu-Gly-Arg-Gly (SEQ ID NO: 5) where the Arg-Gly bond is hydrolyzed, followed by thioredoxin with the V5 epitope displayed at the active site as a surface loop, followed by a GPI linkage to anchor the fusion protein on the cell surface. The other plasmid, designated control, encodes a similar fusion protein without the Factor Xa cleavage sequence.

COS7 cells were electroporated in the presence of either 1 μg or 5 μg plasmid DNA using conditions previously developed at Icogen that result in high levels of fusion protein displayed on the cell surface. The transfected cells were cultured overnight. The medium containing dead, unattached cells was discarded and fresh medium was added to the cells. Cells were cultured for two days post transfection. Because of the possibility that using trypsin to detach cells could result in the cleavage of the Arg-Gly bond, cells were detached using 2 mM EDTA in PBS, pH 7.4, at 37° C. for 20 min. The cells were collected by centrifugation and resuspended in ice-cold medium.

To assess the surface level of fusion proteins, cells expressing each plasmid were incubated on ice with an anti-FLAG monoclonal antibody labeled with biotin for 30 min. The cells were washed 3 times with 1% BSA in PBS, resuspended in this buffer and incubated for 30 min on ice with 2.5 μM magnetic beads coated with streptavidin (Dynal). Cells binding beads were sorted from cells without beads. Both fractions were saved for microscopic analysis to determine the percentage of cells expressing FLAG on their surface. Cells expressing FLAG typically were completely coated with beads. Approximately, 30% of the cells electroporated with 1 μg pIcoFLAGXa or control plasmid and 35-40% of the cells electroporated with 5 μg DNA had high levels of surface FLAG. This is comparable to previous studies performed at Icogen with COS7 cells. FACS analysis was significantly more sensitive in assessing expression levels compared to magnetic bead selection. In earlier studies, it was observed that FACS detects 15 to 20% more positive cells compared to bead selection.

The transfected cells were used to assess the ability of Factor Xa to cleave at the specific sequence Ile-Glu-Gly-Arg-Gly (SEQ ID NO: 5). Cells were washed 3 times in 1% BSA in PBS containing 2 mM calcium chloride. This procedure removed serum protease inhibitors and provided calcium for Factor Xa stability. Cells were incubated for 30 min at room temperature with 1 μg of Factor Xa in 50 μL of buffer. Cells were washed twice in 1% BSA in PBS followed by incubation for 30 min on ice with anti-FLAG antibody labeled with biotin. Cells were washed 3 times in buffer and incubated for 30 min on ice with magnetic beads coated with streptavidin, sorted and examined as described above. None of the cells expressing pIcoFLAGXa were selected by the procedure whereas 30-40% of the cells expressing the control plasmid were selected. The results clearly indicated that the Factor Xa cleavage site within the fusion protein was accessible to proteolysis as predicted. Further, based on the results with cells expressing the control plasmids, the fusion protein was resistant to proteolysis by Factor Xa. Previously, it was observed that similar fusion proteins were not readily digested by trypsin.

### Example 6

### Identification of Peptides that Activate Cell Surface Receptors

This example illustrates the identification of peptides that activate cell surface receptors. Murine and human cell lines have been described and isolated in which cell proliferation is cytokine-dependent. Such cell lines include Ba/F3, AC2 and B9 (see, e.g., Ziegler et al., New Biol. 3:1242-48 (1991)). Ba/F3 cells are cytokine-dependent and only grow in the presence of IL-3. The factor-dependence can be overcome if the cells carry the cytoplasmic signaling domain of one of many receptors, including c-kit (Jin et al., Blood 91:890-97 (1998)), Epo (Blau et al., Proc. Natl. Acad. Sci USA 94:3076-81 (1997)), c-mpl (Jin et al., supra), and JAK2 kinase (Mohi et al., Mol. Biol. Cell 9:3299-308 (1998)). Dimerization of the cytoplasmic domains is required to circumvent the cytokine requirement. Dimerization of binding domain-cytoplasmic domain chimeric fusions can be achieved through the use of chemical inducers, which cross-link the binding domains.

Alternatively, models are available to demonstrate that peptides displayed on the cell surface can be used to search for biologically active peptides that target various receptors on either the host cell or other cells. Experiments were conducted using the BaF3/thrombopoietin receptor (TPO-R) cell line, an IL-3 dependent cell line with TPO dependence conferred by the expression of recombinant TPO receptor within these cells (Lok et al., Nature 369:565-68 (1994)). Peptides have been previously identified that activate the TPO receptor (Cwirla et al., Science 276:1696-99 (1997)).

A nucleic acid encoding one of the TPO receptor binding peptides, mpl, was inserted between nucleic acid sequence encoding CD24 secretory sequence, a nucleic acid encoding thioredoxin, and the CD24 sequence for GPI linkage in an expression vector. This vector and a control vector (without a nucleic acid encoding mpl) were electroporated into separate cultures of BaF3/TPO-R cells. The cells were grown in the presence of IL-3 for three days to allow for expression of the TPO mimetic peptide. IL-3 was then removed from the cultures, and the cells were further cultured. No factor-independent growth of cells developed from 2 million cells transfected with the control plasmid.

In contrast, cells transfected with the plasmid encoding, mpl, the TPO mimetic, sustained factor-independent growth of cells for several weeks. The survival of these cells indicated the utility of this vector for screening random peptide libraries for specific peptides that activate cell surface receptors/proteins on host and other cells. This example can be repeated in the BaF3/TPO-R cell line following transfection of the cells with the surface tethered random peptide libraries described in Example 4.

### Example 6

### Identification of Peptides that Affect Apoptosis in Cells

The results of Examples 4 and 5 indicate that peptide libraries also can be used to identify peptides that increase or decrease apoptosis (i.e., programmed cell death) in cells via specific interaction with surface receptors that influence apoptosis. This example utilizes a well-known assay to detect desired cells shortly after apoptosis has been activated. Minutes following stimuli to initiate apoptosis, cell membrane phospholipid phosphatidylserine (PS) is translocated from the inner leaflet of the membrane to the outer one. The loss of cell membrane asymmetry precedes the intracellular reactions. Exposed to the extracellular environment, PS can be detected on living cells by a 35 kDa, calcium-dependent, phospholipid-binding protein, Annexin V (see, e.g., Raynal and Pollard, Biochim. Biophys. Acta 1197:63-93 (1994)). Annexin V has high specificity for PS and is readily labeled with fluorophores, thereby providing the basis of selective assays (e.g., FACS analysis) to detect apoptotic cells.

Annexin V will also bind to dead cells displaying PS. To identify live cells, the cells can be screened with the vital dye, propidium iodide (PI) or 7-amino-actinomycin D (7-AAD). Cells induced into apoptosis will eventually die and bind both reagents. Thus, the apoptotic state of cells is typically determined shortly after contacting with the peptide library. Screening assays can be used to select Annexin V-positive, PI-negative cells that represent cells in the earliest stage of apoptosis before DNA fragmentation has occurred.

The population of cells collected with the desired phenotype (i.e., Annexin V-positive, PI-negative) in the first round of selection will also contain a variety of background cells including those retaining some apoptotic potential, cells damaged due to manipulation, cells containing host mutations promoting apoptosis, and cells expressing surface peptides that may bind Annexin V. Most of these cells and other types of background cells will be eliminated in later rounds of selection.

Specific peptide interaction with Annexin V might present a slight problem because the screening assay cannot distinguish such an interaction from early stage apoptotic cells. However, further observation can demonstrate that cells binding Annexin V directly via surface peptides do not progress down the apoptotic pathway and die. A similar problem can arise if a peptide's activity is limited to phosphatidylserine exposure to the cell exterior without significant increases in apoptosis.

In addition, expression of presentation molecules and some library peptides may result in some degree of cytotoxicity. However, general peptide library toxicity appears to be low based on the results of experiments using the fluorescent tracking dye, PKH 26 (see, e.g., Horan et. al., Methods Cell Biol. 33:469-90 (1990)). Also, apoptosis is a regulated process distinct from necrosis (Kerr et al., Br. J. Cancer 26:239-57 (1972)), and assays are available to determine whether peptides are targeting apoptosis or blocking important cell pathways thereby causing death. To distinguish these interactions from apoptosis-inducing library peptides, peptides are screened, for example, by direct microinjection into cells or administration to cells in vivo or ex vivo. (See, e.g., Hollinger et al., J. Biol. Chem. 274:13298-304 (1999).)

Other assays can be employed to confirm that Annexin V-positive cells are committed to apoptosis. Assays to detect DNA fragmentation using FACS analysis are available to select cells at any time-point (see, e.g., Li and Darzynkiewicz, Cell Prolif. 28:572-79 (1995); Li et al., Cytometry 20:172-80 (1995)). Typically, apoptotic cells can be detected with these reagents 2 to 12 hours after treatment with anti-Fas antibody. Because so many cells must be screened during the first round of selection, these additional assays will be more practical if used in subsequent rounds of selection.

Several controls can be performed to verify that the screening assay is operating as expected. Negative controls can include, for example, host cells transfected with a vector encoding the presentation protein alone and/or a neutral clone (i.e., not affecting apoptosis) from the peptide library. Positive controls can include, for example, cells induced into apoptosis by a monoclonal antibody to Fas (BD PharMingen, San Diego, Calif.), Granzyme B, serum deprivation, hypoxia, or chemical agents such as doxorubicin, TGFβ1, cisplatin or camptothecin (Maccarrone et al., Eur. J. Biochem. 441:297-302 (1996); Johnson et al., Leuk. Res. 21:961-72 (1997)). Also, unlabeled Annexin V can be included in some experiments as a control for the specificity of the staining protocol.

High-speed FACS instruments can be used to process large numbers (e.g., hundreds of millions) of independent transfectants per day. Alternatively, magnetic bead selection can be used to facilitate first round screening. Preliminary experiments with beads have confirmed the feasibility of this approach using COS7 or K562 cells expressing a cell surface library containing FLAG. Two types of magnetic beads conjugated with streptavidin were tested, 2.8 micron beads (Dynal, Lake Success, N.Y.) and 50 nanometer beads (Miltenyi Biotech, Germany). In one demonstration, anti-FLAG antibody labeled with biotin was incubated with untransfected COS7 cells spiked with varying numbers of COS7 cells expressing cell surface tethered thioredoxin with a FLAG marker. The Miltenyi system involves column selection, while the Dynal beads are captured with a large magnet. Each system is capable of processing billions of cells in a few hours. The Miltenyi method requires more time, however. Each system has a low background of non-specific selection of approximately 0.002% or 1 out of 50,000 cells, without any attempt to optimize the procedures.

Bead selection can greatly reduce the number of background cells prior to FACS analysis. The preselected cells can then be sorted by FACS. While bead selection cannot distinguish between live and dead cells, FACS can identify Annexin V positive and PI negative cells for further enrichment. Initially, it can be estimated that the frequency of positive cells in a first round of selection will be approximately 1 in about 6 million cells, although greater and lesser frequencies are possible.

From the initial round of selection, or from subsequent rounds of selection, expression library DNA can be isolated from the desired selected cells by, for example, Hirt lysis method to form a sub-library (see, e.g., Hirt, J. Mol. Biol. 26:365-69 (1967); Sambrook et al., supra; Ausubel et al., supra). The sub-library can be amplified in an E. coli host strain, and then DNA from that sub-library can be reintroduced into a mammalian cell line for subsequent rounds of enrichment.

The complexity of the plasmid population of each sub-library can be assessed after each round of selection or enrichment. Briefly, the complexity of the library can be assessed by determining whether randomly selected expression library oligonucleotides hybridize to dilutions of the expression library DNA. Serial dilutions of the original library and/or sub-libraries can be made on a suitable hybridization membrane. For example, successive 1:10 dilutions of expression library DNA can be applied to a hybridization membrane using a multi-well apparatus. Randomly selected expression library clones can be linearized (e.g., at the 5′ restriction endonuclease site), and radioactively labeled RNA can be transcribed from a priming site. The radiolabeled RNA can be used to probe the serial dilutions of the library to determine the degree of hybridization.

When the sub-libraries reach a sufficiently low level of complexity, the individual expression library vectors can be introduced into the mammalian host cells to confirm the activity of peptides. In some cases, the positive expression library clones can be further tested in other human cell lines (e.g., HepG2 cells) to confirm that the peptides are generally effective against other cell types.

The library oligonucleotides from the confirmed positive expression library clones can be subjected to DNA sequence analysis to determine the sequence of the encoded peptide. If there are a number of peptides that express activity, it will be useful to determine if those peptides affect the same or different target molecules. The identity of the targets can be determined, for example, using ¹²⁵I-labeled peptides. Different peptides can be compared to determine whether the targets are the same or different. Suitable fractionation procedures can include, for example, Western blot analysis, two-dimensional electrophoresis, column chromatography, FPLC and/or HPLC. (See generally Ausubel et al., supra; Sambrook et al., supra.)

Effective peptides are expected to have specificity commensurate with high binding affinity toward target molecules. Thus, peptides attached to a fusion partner can be used as affinity ligands to purify targets. Several options are available for determining partial amino acid sequence data, including mass spectrometry analysis and micro-protein sequencing, for identifying a target when a sufficient quantity of target has been purified. The sequence of a known or unknown target proteins can be used to search a public database, such as GenBank, to ascertain its identity.

Screening of cells expressing surface peptides can readily identify agonists of trophic factors that suppress apoptosis via known or unknown receptors. For example, K562 cells rapidly enter apoptosis in the absence of serum, with the addition of TGFβ1 or cisplatin (see, e.g., Maccorrone et al., Eur. J. Biochem. 241:297-302 (1996)). Transfected K562 cells expressing peptides cultured in the presence of pro-apoptotic agents can be screened within hours or a few days at most for non-apoptotic cells among mostly dead or dying cells. This procedure can provide a major advantage for identifying trophic peptides. In contrast, it is difficult early in traditional growth selection approaches to identify a few viable cells among hundreds of millions of dying ones. A slight modification of this procedure also has utility in identifying antagonists of trophic factors and that should be pro-apoptotic.

### Example 7

### Epitope Mapping of Antibodies

The methods according to the present invention can be used to determine the epitopes of antibodies under physiologic conditions. To demonstrate this application of the methods according to the present invention, two experiments were initiated. First, 400 million COS7 cells were transfected 3.5 days earlier with a peptide library containing random 12 residue long peptides in linear array at the N-terminus of a presentation molecule. The COS7 cells were screened with a monoclonal antibody to polyhistidine, 6Xhistidine (Sigma, St. Louis, Mo.), to identify those cells expressing this epitope in the library. The prediction was that a minimum of 40 cells would express the epitope, but many more positive cells were possible if 5Xhistidine sequences also were cross-reactive with the antibody. Following treatment with the polyhistidine antibody, the cells were incubated with rabbit polyclonal anti-mouse antibody labeled with biotin, and then any positive cells were selected on magnetic beads coated with streptavidin. Non-transfected COS7 cells were used in control experiments. In the first round of screening, approximately 2,000 cells were selected from the transfected and from the control cells. No enrichment of transfected cells over control cells was expected in the first round of screening.

The DNA was recovered and treated as described in Example 6 and transfected into naïve COS7 cells. Four days following transfection, a second round of screening for the polyhistidine epitope was conducted. In this round, between 0.002% and 0.003% of the total control cells were selected on magnetic beads, whereas 0.3% (approximately 100 times more cells) of the total transfected cells were selected on beads. The procedures were repeated, and a third round of screening was conducted. Again, the same percentages of total cells were selected, indicating no further enrichment of transfected cells over control cells. A fourth round of screening resulted in no enrichment of selected transfected cells over control cells.

In the second experiment using 2.46×10⁸ transfected K562 cells, a mouse antibody recognizing the epitope Tyr-Gly-Gly-Phe-Leu was employed for screening. The cells were incubated with an anti-mouse antibody labeled with biotin, followed by selection of magnetic beads. The procedures were repeated for 4 additional rounds, and the percentage of total cells recovered in each round were as follows: round 1, 0.00024%; round 2, 0.0018%; round 3, 0.12%; round 4, 0.075%; and round 5, 0.028%.

In both experiments, the same results were observed in the first 2 to 3 rounds of screening followed by little further enrichment in subsequent rounds. Examination on gels of the DNA recovered from each round of COS7 cell screening revealed that by the second round a considerable degree of plasmid degradation had occurred. By the fourth round, there was little intact plasmid observed.

Two processes contributed to the extensive degradation of plasmid DNA. First, plasmids were degraded in COS7 cells over the 10 to 12 days of total culture time. Second, the vector design can lead to selective amplification of plasmid fragments during the transformation in bacteria. To avoid degradation, high levels of presentation molecules can be expressed just one day following transfection and, thus, it is not necessary to culture cells 3 to 4 days in each round before screening them. Many more intact plasmids can be recovered with shorter screening times, resulting also in considerable time savings. Additionally, following each round of selection, the peptide inserts from recovered DNA can be PCR amplified and ligated into unused vector without inserts prior to transformation in bacteria.

### Example 8

### Identification of Peptides that Mimic Antigenic Binding Sites of Autoantibodies

Peptide(s) that bind to antigen binding sites, but have amino acid sequences different from natural antigens, so-called mimotopes, can be effective competitive inhibitors of autoantibody binding. Mimotopes frequently can be identified in constrained loop arrays of peptides (see, e.g., McConnell et. al., Gene 30:115-18 (1994)) and, therefore, screening experiments can use these types of libraries in addition to those expressing peptides in linear array. However, specific peptide sequences present in naturally-occurring antigens that bind to autoantibodies are more likely to be identified in linear arrays of peptides. A few of the available autoantibody targets that can be used in screens include lupus (antibodies to nuclear antigen), diabetes (glycated hemoglobin, glycated albumin), rheumatoid arthritis (rheumatoid factor), thyroid disease, such as, for example, Graves' disease (thyroglobulin) and heparin-induced thrombocytopenia (HIT) (heparin-platelet factor 4).

For example, peptides that specifically bind the antigenic binding site of rheumatoid factor (RF) can be identified by detecting RF-binding to the surface of host cells expressing peptides in linear or constrained loop arrays, followed by isolation of RF-binding cells by FACS sorting. RF, which is generally IgM, can be purified by conventional affinity purification techniques (see Harlow and Lane, supra). A peptide library can be expressed in non-adherent host cells, such as, for example, CHO or K562 cells, and these peptide-expressing cells then exposed to the affinity purified RF in undiluted blood, plasma, serum or synovial fluid depleted of human IgG, the natural RF antigen, at 37° C. IgG can be removed from blood preparations, for example, by adsorption to immobilized Protein A. Suitable concentrations of RF for screening live cells can be predetermined by testing various concentrations of RF against the Fc region of human IgG expressed on CHO or K562 cells using, for example, the same expression vector as used for the peptide library. (Similar methods can be used to characterize selection assays and to distinguish between high-quality and poor peptides). Following centrifugation of host cells at low speed, a wash in RF-free and IgG-free blood, plasma, or serum, and final resuspension in RF-free and IgG-free blood, plasma, or serum, host cells can be incubated with a fluorescently-labeled anti-IgM antibody. RF-bound cells can then be detected and sorted, for example, into a microtiter plate by FACS. These isolated, RF binding (positive) cells can then be expanded and retested against RF to determine those cell populations that exhibit high affinity binding. The peptide expression vectors can then be isolated from these identified clones to build a sub-library of RF-binding peptides and the peptide sequences determined.

### Example 9

### Identifying Peptides that Bind Soluble Factors Involved in Disease

This example demonstrates the utility of the present invention in the identification of peptides that specifically bind to tumor necrosis factor-α (TNF), a soluble protein involved in diverse immunological and inflammatory disorders. The studies using TNF provide a model for other soluble factors found in or secreted from humans, animals, parasites, and microorganisms. Screens can include TNF and K562 cells expressing peptide libraries on their surface in various media including, for example, undiluted blood, plasma or serum. The K562 cells, which can be fixed in para-formaldehyde prior to screening, are derived from a human leukemia and, therefore, are compatible with screening procedures in blood preparations. Specific anti-TNF antibodies labeled with biotin and a fluorophore can be included in the screens. K562 cells can be pre-selected prior to analytical selection by using several procedures. For example, when screens are conducted in undiluted blood, it can be useful to adsorb all K562 cells to magnetic beads by taking advantage of a selectable marker such as FLAG. The pre-selection step can eliminate blood cells from further screening. A pre-selection step using magnetic beads also can be used to collect all cells binding TNF. Since some blood cells can bind TNF, the addition of anti-FLAG labeled with a second fluorophore prior to FACS analysis can permit the recovery of cells expressing FLAG and binding TNF. Magnetic bead selection used for all of the screening steps can be an alternative to FACS.

DNA from selected cells can be recovered and processed as described infra. Further rounds of screening can be performed to enrich for peptides that bind to TNF. When peptides binding to TNF with high avidity are identified, assays known to those skilled in the art can be used to determine which peptides effectively block the binding of TNF to TNF-receptors on cells. Such peptides are also candidates for further development into therapeutic agents.

### Example 10

### Identification of Peptides that Bind to Proteins and Other Macromolecules on the Surface of Cells Parasites. Microorganisms. Viruses and Isolated Subcellular Organelles

The methods according to the present invention can also be used for the identification of peptides that bind to unique proteins and other macromolecules expressed on the surface of cells, parasites, viruses and subcellular organelles. Such screening can be conducted, for example, under physiological conditions, such as undiluted blood, plasma, or serum at 37° C., the environment in which proteins and other macromolecules on mammalian cells, parasites and viruses can express their activities.

To demonstrate this method of the present invention, it can be established that certain peptides within the surface display libraries can bind with high avidity to target molecules on other cells. Two exemplary protocols can be done to demonstrate such binding. First, surface peptide libraries can be expressed on K562 cells and incubated with isolated mammalian blood platelets reconstituted in plasma. Centrifugation at 100×g will pellet the K562 cells while leaving most of the platelets in the supernatant fluid. The cell pellet can be resuspended in fresh medium, and the cells pelleted again by centrifugation. The process can be repeated several times to remove as many of the unbound platelets as possible.

Several platelet specific antibodies are available. These antibodies when labeled with a fluorophore can be used with antibody (labeled with a second fluorophore) as a marker on K562 cells to collect by FACS only cells associated with both fluorophores. Repeated rounds of selection can enrich for peptides that bind to molecules on the surface of platelets. Platelets are such specialized blood elements that there will be a high probability of identifying peptides that bind to a platelet-specific protein or other macromolecule.

In the second protocol, peptides are identified that bind to cell surface molecules and that bind to unique surface target molecules. The cassette design of the expression vector permits the quick removal or addition of individual elements in constructs. A presentation molecule (PM-1) can be constructed containing native green fluorescent protein (GFP) and the elements needed for secretion and tethering of PM-1 to the cell surface (but lacking the elements encoding the peptide library) and either the FLAG or V5 epitope. GFP is not a surface molecule of COS7 or K562 cells and, thus, expression of PM-1 introduces a unique cell-surface target protein on these cells. (GFP itself is an excellent marker, but the presence of an additional marker in the construct can be useful.) Another presentation molecule (PM-2) can be constructed that contains all of the elements of the existing surface peptide libraries and the epitope marker not encoded in PM-1.

Transfecting millions or billions of K562 cells with DNA encoding PM-2, followed by fixing the cells in para-formaldehyde can provide a large diverse peptide library that can be used repeatedly. DNA encoding PM-1 can used to transfect K562 cells to provide target cells in the screen. A few million target cells can be sufficient for screening. Alternatively, libraries can be displayed on K562 cells, and target cells can be prepared from COS7 cells. The levels of target GFP on the cell can be varied to help determine the specificity of binding. Control experiments can be conducted using cells transfected with a presentation molecule lacking GFP. Screening can be performed in a variety of physiologic fluids, such as, for example, undiluted blood, plasma or serum at 37° C.

The previous examples are provided to illustrate but not to limit the scope of the claimed inventions. Other variants of the inventions will be readily apparent to those of ordinary skill in the art and encompassed by the appended claims. All publications, patents, patent applications and other references cited herein are hereby incorporated by reference.

1. A method for identifying peptides that specifically bind to extracellular target molecules, comprising:
introducing an expression library comprising a plurality of oligonucleotides, at least a majority of the oligonucleotides having different sequences encoding different peptides, into a first plurality of mammalian host cells, the host cells expressing and displaying the peptides on an extracellular cell surface;
contacting the host cells displaying the peptides with at least one extracellular target molecule under substantially physiological conditions;
selecting from the first plurality of host cells displaying the peptides a first subset of cells displaying peptides that bind to the at least one target molecule, and
recovering from the first subset of host cells a first sub-library of the expression library comprising at least one oligonucleotide that encodes a peptide that binds to the at least one target molecule.

2. The method of claim 1, wherein the contacting is performed in the presence of a complex biological fluid.

3. The method of claim 2, wherein the complex biological fluid is blood, serum, plasma, sweat, tears, urine, semen, vaginal fluid or mucous.

4-6. (canceled)

7. The method of claim 1, further comprising:
introducing the first sub-library into a second plurality of host cells, the second plurality of host cells expressing peptides encoded by the first sub-library and displaying the peptides on the extracellular cell surface;
contacting the second plurality of host cells displaying the peptides with the at least one extracellular target molecule;
selecting from the second plurality of host cells a second subset of host cells displaying peptides that bind to the at least one target molecule; and
recovering from the second subset of selected host cells a second sub-library comprising at least one oligonucleotide that encodes the peptide that binds to the at least one target molecule.

8. (canceled)

9. The method of claim 1, wherein the target molecule is an extracellular protein, a carbohydrate, or a lipid.

10. The method of claim 9, wherein the extracellular protein is a peptide, protein, antibody, glycoprotein, phosphoprotein, glycophosphoprotein, proteoglycan, or a polymeric complex thereof.

11-12. (canceled)

13. The method of claim 1, wherein the target molecule is displayed on an extracellular surface of a target cell.

14. The method of claim 13, wherein binding of the peptide to the target molecule results in a change in a detectable phenotype of the target cell.

15. The method of claim 14, wherein the change in detectable phenotype is
(a) a change resulting from altering a function of a mutant protein; (b) an alleviation of factor-dependent growth; or (c) a change in apoptotic state of the target cell.

16-17. (canceled)

18. The method of claim 1, wherein the target molecule is displayed on an extracellular surface of an animal cell.

19. The method of claim 18, wherein the animal cell is a mammalian cell.

20. The method of claim 18, wherein the selecting comprises
contacting the animal cell with a detectably labeled antibody that binds a marker on the animal cell and detecting the bound, labeled antibody.

21. The method of claim 18, wherein the selecting comprises contacting the host cells with a detectably labeled antibody that binds a marker on the host cells and detecting the bound, labeled antibody.

22-23. (canceled)

24. The method of claim 1, wherein the target molecule is (a) a molecule that binds to a cell surface receptor; (b) an antibody, wherein the binding of the peptide to the antibody reduces binding of the antibody to an antigen; (c) an extracellular enzyme, wherein the binding of the peptide to the enzyme alters the activity of the enzyme; or (d) a tumor-specific antigen or a tumor-associated antigen.

25. The method of claim 24, wherein the target molecule is a cytokine, a chemokine, or a secreted factor; and the binding of the peptide to the target molecule, alters binding of the target molecule to the cell surface receptor.

26. The method of claim 25, wherein the binding of the target molecule to the cell surface receptor is reduced.

27. (canceled)

28. The method of claim 24, wherein the antibody is an autoantibody.

29. (canceled)

30. The method of claim 24, wherein (i) the peptide binds to an active site of the extracellular enzyme, thereby inhibiting activity of the enzyme; or (ii) the peptide binds to an allosteric regulatory site on the extracellular enzyme.

31. The method of claim 24, wherein the peptide is a competitive inhibitor of substrate binding to the extracellular enzyme.

32-33. (canceled)

34. The method of claim 1, wherein the target molecule is displayed on an extracellular surface of a target cell.

35. The method of claim 34, wherein the target molecule is a mutant protein, and binding of the peptide alters a function of the mutant protein.

36. The method of claim 34, wherein peptide binding alleviates factor-dependent growth; changes an apoptotic state of the target cell; or causes increased or decreased sensitivity to a cytotoxic drug.

37-38. (canceled)

39. The method of claim 1, wherein the peptides are displayed as a fusion protein with a presentation molecule.

40. The method of claim 39, wherein the presentation molecule is CD24 or IL-3.

41. (canceled)

42. The method of claim 39, wherein the fusion protein further comprises an epitope.

43. The method of claim 42, wherein the epitope is polyhistidine, V5, FLAG, or myc.

44. The method of claim 39, wherein the fusion protein further comprises a signal for a glycophosphatidylinositol anchorage.

45. The method of claim 1, wherein the selecting comprises use of a flow sorter to identify the first subset of cells exhibiting binding to the target molecule.

46. The method of claim 34, wherein the target cells are coupled to magnetic beads and the selecting comprises collection of the first subset of cell bound to the target cells.

47. The method of claim 1, wherein the target molecule is detectably labeled and the selecting comprises identifying host cells bound to the labeled target molecule.

48. The method of claim 1, wherein the target molecule comprises detectably labeled antibody and the selecting comprises identifying host cells bound to the labeled antibodies.

49. The method of claim 1, wherein the target molecule is a protein associated with an autosomal dominant disease, with an oncogenic disease, or with normal cellular function.

50. A peptide display library, comprising:
a plurality of at least one type of expression vector, each expression vector having
a first nucleic acid sequence encoding a signal sequence, a presentation molecule comprising modified CD24 or modified IL-3 receptor, and a transmembrane domain, and
a cloning site for insertion of a second nucleic acid sequence distal to the transmembrane domain, the second nucleic acid sequence encoding an amino acid sequence;
whereby fusion proteins are expressed and displayed on an extracellular surface of a host cell.

51. The library of claim 50, wherein the second nucleic acid sequence encodes peptides having up to 20 amino acids.

52. A library peptide displayed as an extracellular membrane protein fusion, comprising:
modified CD24 comprising a signal sequence, a library peptide inserted within CD24 amino acid sequence, and a transmembrane domain;
whereby the peptide is displayed on an extracellular cell surface such that the peptide can interact with extracellular target molecules under substantially physiological conditions.

53. A plasmid expression vector, comprising:
an SV40 origin of replication; and
a nucleic acid sequence comprising SV40 early promoter region and SV40 Large T antigen coding region, the SV40 early region promoter promoting transcription of the Large T antigen coding region;
whereby replication of the plasmid is self-regulating.

54-68. (canceled)

69. The method of claim 2, wherein the complex biological fluid is substantially undiluted.
